(19) United States
(12) Patent Application Publication
(10) Pub. No.: US 2018/0002379 A1
(43) Pub. Date: Jan. 4, 2018

(54) METHODS AND COMPOSITIONS FOR IDENTIFICATION OF HIGHLY SPECIFIC NUCLEASES

(71) Applicants: Sangamo Therapeutics, Inc., Richmond, CA (US); The Trustees of Princeton University, Princeton, NJ (US)

(72) Inventors: Jeffrey C. Miller, Richmond, CA (US); Marcus B. Noyes, Princeton, NJ (US)

(21) Appl. No.: 15/543,516

(22) PCT Filed: Jan. 21, 2016
(86) PCT No.: PCT/US2016/014283

§ 371 Date: Jul. 13, 2017

Related U.S. Application Data
(60) Provisional application No. 62/106,042, filed on Jan. 21, 2015.
(60) Provisional application No. 62/212,805, filed on Sep. 1, 2015.

(51) Int. Cl. C07K 7/06; C07K 14/805; C12N 9/22

(52) U.S. Cl. CPC C07K 7/06; C12N 9/22; C07K 14/805; C07K 2319/81

(57) ABSTRACT

Disclosed herein are methods and compositions for identification of specific DNA binding domains for constructing highly specific nucleases, which allows for pristine genome editing.

### CROSS-REFERENCE TO RELATED APPLICATIONS

The present application claims the benefit of U.S. Provisional Application No. 62/106,042, filed Jan. 21, 2015 and U.S. Provisional Application No. 62/212,805, filed Sep. 1, 2015, the disclosures of which are hereby incorporated by reference in their entireties.

### STATEMENT OF RIGHTS TO INVENTIONS MADE UNDER FEDERALLY SPONSORED RESEARCH

Not applicable.

### TECHNICAL FIELD

The present disclosure is in the fields of gene modification, particularly identification of highly specific nuclease for targeted genomic modification.

### BACKGROUND

Gene modification holds enormous potential for a new era of human therapeutics. These methodologies will allow treatment for conditions that have not been readily addressable by standard medical practice.

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, e.g., U.S. Patent Nos. See, e.g., U.S. Pat. Nos. 9,045,763; 9,005,973; 8,956,828; 8,945,868; 8,697,359; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983 and 20130177960 and 20150056705; U.S. application Ser. No. 14/706,747 and Swarts et al (2014) Nature 507(7491): 258-261), the disclosures of which are incorporated by reference in their entireties for all purposes. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or insertion of a sequence of interest (targeted integration). This technique can also be used to introduce site specific changes in the genome sequence through use of a donor oligonucleotide, including the introduction of specific deletions of genomic regions, or of specific point mutations or localized alterations (also known as gene correction). Cleavage can occur through the use of specific nucleases such as engineered zinc finger nucleases (ZFN), transcription-activator like effector nucleases (TALENs), or using the CRISPR/Cas system with an engineered crRNA/tracr RNA (single guide RNA′) to guide specific cleavage. Targeted nucleases based on the Argonaute system (e.g., from T. thermophilus, known as ‘TtAgo’, see Swarts et al (2014) Nature 507(7491): 258-261) may have the potential for uses in genome editing and gene therapy.

As applications for designer nucleases have multiplied and extended into more sensitive areas such as stem cell biology and therapeutics, the focus of design efforts have shifted from improving activity to optimizing genome-wide specificity. A key element of this shift has been the emerging realization that nucleases designed for genome editing applications will frequently need to discriminate against highly similar off-target sequences, due to the high frequency of repeat elements, gene duplications and pseudogenes in eukaryotic genomes. Further complicating matters, successful HDR can be limited by the distance between the DSB and the sequence to be modified. For these reasons, the precise location of a DSB may be critical and similar off-target sequences unavoidable.

At the crux of a high-fidelity designer nucleases are the abilities to both strongly specify a desired sequence and the ability to avoid others. Strong in vivo, on-target activity has been demonstrated for all the designer nuclease platforms (zinc finger nucleases (ZFNs), meganucleases, transcription activator-like effector domains (TALENs), and RNA guided CRISPR/cas9 systems (RGENs)). See, e.g., U.S. Patent Publication No. 20090111119. However, some systems, such as the CRISPR/Cas system, exhibit substantial activity at alternative off target loci similar to the designed target. See, e.g., Fu et al. (2013) Nat Biotechnol. 31(9):822-6.

Thus, there remains a need for methods and compositions for identifying DNA binding domains of high specificity that distinguish between two closely related sequences as well as for novel DNA binding domains (e.g., identified using these methods).

### SUMMARY

Disclosed herein are methods and compositions that identify DNA binding domains that discriminate between highly homologous sequences and thus can be fused to a nuclease domain to target only a single specific sequence. In particular, a multi-reporter selection system is disclosed to screen libraries of DNA binding domains that will differentially interact with one target DNA over another. The selected DNA binding domains can then be fused to a nuclease domain to develop engineered nucleases that can discriminate between two similar targets while exhibiting strong on-target activity (cleavage) with minimal or no detectible cleavage activity at highly homologous off-target sequences.

The present disclosure relates to identification of highly-specific DNA binding domains, for example DNA-binding domains used in engineered TALEs, Cas/guide RNA combinations, meganuclease binding domains and/or zinc finger proteins (ZFPs). Specifically, the methods and compositions described herein allow for the identification of DNA binding domains that distinguish between two highly homologous target sites.

In one aspect, described herein is a reporter construct for identifying highly specific DNA binding domains, including identifying DNA binding domains that distinguish between at least two similar target sites. The reporter construct comprises two or more target sites for a DNA binding domain, for example two or more similar paired target sites. In certain embodiments, the construct comprises two, three, four or more different target sites. Preferably, the target sites are different from each other. At least one reporter is linked to each of the target sites and the reporters at each target site are different from each other so that binding at each target site can be assessed individually using the reporter associated with that site. Furthermore, expression of each reporter is driven by a separate promoter such that activity of the DNA binding domain on each of the multiple target sites can be assayed independently. The multiple promoters (e.g., first and second promoters in the case of two target sites) may be the same or may be different. In addition, the multiple promoters may be constitutive, inducible, strong or weak. The reporter genes may encode selectable (positive and/or negative) markers, for example His3 and/or Ura3; and/or detectable reporters such as one or more fluorescent proteins (e.g., green or red). In certain embodiments, the reporter comprises at least two reporters operably linked to one or more of the plurality of target sites. Expression of the at least two different reporters in a host cell results in a signal that is measurable by suitable assays (and/or selection), for example by colorimetric or enzymatic assays performed on intact or lysed cells. In certain embodiments, activity of the reporter gene is determined by assaying levels of a secreted protein (e.g., the product of the reporter gene itself or a product produced directly or indirectly by an active reporter gene product). In certain embodiments, the reporter construct comprises a construct as shown in FIG. 1B.

In another aspect, described herein is a host cell (or population of host cells) comprising any of the reporter constructs described herein. The host cell typically is a prokaryotic cell that can be transformed with a library of putative DNA binding domains. In some aspects, the prokaryotic cell is an E. coli cell. The reporter construct may be transiently maintained in the host cell. Alternatively, the reporter construct is stably integrated into the genome of the host cell.

In yet another aspect, methods of identifying a DNA-binding domain that binds to a specific target site are provided. In certain embodiments, the methods comprise introducing one or more DNA binding domains and/or one or more DNA binding domain-expression constructs encoding one or more DNA binding domains into a host cell comprising a reporter construct as described herein, the reporter construct comprising a target sequence recognized by the DNA binding domain(s); incubating the cells under conditions such that the DNA binding domain(s) are expressed; and measuring the levels of reporter gene expression in the cells, wherein increased levels of reporter gene expression are correlated with increased binding of the DNA binding domain and the target sequence. The DNA binding domain may comprise, for example, a non-naturally occurring DNA-binding domain (e.g., an engineered zinc finger protein or an engineered DNA-binding domain from a homing endonuclease) or a natural DNA binding domain. In certain embodiments, the DNA-binding domain is in a nuclease and the methods comprise identifying a nuclease that cleaves the specific target site bound by the DNA-binding domain(s).

In yet another aspect, methods of identifying a DNA binding domain that distinguishes between two similar target sites are provided. The methods comprise introducing a DNA binding domain and/or expression constructs encoding the DNA binding domain into a host cell comprising a reporter construct as described herein (e.g., a reporter construct comprising similar target sites); incubating the cells under conditions such that the DNA binding domain(s) are expressed; measuring the levels of the first and second reporter gene expression in the cells; and determining the DNA binding domain that preferentially targets to one target site (e.g., by assaying reporter gene levels associated with each target site). In certain embodiments, less than 1% of the similar target site(s) are bound by the DNA binding domain (e.g., less than 0.5%, less than 0.1%). The DNA-binding domain may be part of a fusion protein or nuclease system, for example, a fusion of a DNA-binding domain and regulatory domain such as a cleavage domain.

In another embodiment, the invention comprises methods to identify highly specific guide RNA/Cas DNA binding domain combinations. The methods comprise introducing a nuclease defective Cas protein and/or an expression construct encoding the nuclease defective Cas protein into the host cell comprising the reporter construct as described herein, and then further introducing potential guide RNAs or expression constructs encoding the potential guide RNAs. The cells are then incubated such that the nuclease defective Cas protein and guide RNAs are expressed and then reporter expression is analyzed. The guide RNA is identified with the highest differential between expression at the desired target versus the off target sequence. This guide is then used with a CRISPR/Cas system such that the desired target is preferentially cleaved as compared to the non-desired target.

Also provided are DNA-binding domains, including, for instance DNA-binding domains identified by any of the methods described herein. In certain embodiments, the DNA-binding domain binds to a target site as shown in Table A (SEQ ID NOs:28-33, 66, 94, 127, 128, 129 or 142 in an Hbb or CCR5 gene. In other embodiments, the DNA-binding domain comprises a zinc finger protein comprising the recognition helix regions in the order and sequence shown in Table A.

In another aspect, described herein is a genetically modified cell or cell line, for example as compared to the wild-type sequence of the cell or cell line. Wild-type Hbb sequences (e.g., without mutations) are known in the art, for example as described in Taylor et al. (2006) Nature 440(7083): 497-500 and GenBank Accession No. NC_000011 (bases 1 to 1,606 show a wild-type Hbb sequence). In certain embodiments, the cells comprise genetically modified red blood cell (RBC) precursors (hematopoietic stem cells known as “HSCs”). The cell or cell lines may be heterozygous or homozygous for the modification. The modifications may comprise insertions, deletions and/or combinations thereof. In certain embodiments, the HSCs are modified with an engineered nuclease and a donor nucleic acid such that a wild type gene (e.g., globin gene) is inserted and expressed and/or an endogenous aberrant gene is corrected. In certain embodiments, the modification (e.g., insertion and/or deletion) is at or near the nuclease(s) binding and/or cleavage site(s), for example, within 0-300 (or any value therebetween) base pairs upstream or downstream of the site(s) of cleavage and/or binding sites, more preferably within 0-100 base pairs (or any value therebetween) of either side of the binding and/or cleavage site(s), even more preferably within 0 to 50 base pairs (or any value therebetween) on either side of (and/or including one or more bases within) the binding and/or cleavage site(s). In some embodiments, the modification is at or near a genomic sequence as shown in the first column of Table A (e.g., at or near SEQ ID NOs:28-33, 66, 94, 127, 128, 129 or 142) and may include a modification at the binding and/or cleavage site(s), namely modification of one or more of the nucleotides within the binding site of the DNA-binding domain and/or cleavage site of the nuclease. In some cases, the wild type gene sequence for insertion encodes a wild type β globin. In other cases, the endogenous aberrant gene is the 13 globin gene, for example one or more genomic modifications that correct at least one mutation in an endogenous aberrant human beta-hemoglobin (Hbb) gene. Cells descended from the cells or cell lines as described herein are also provided, including but not limited to, partially or fully differentiated cells descended from modified stem cells as described herein (e.g., RBCs or RBC precursor cells). Compositions such as pharmaceutical compositions comprising the genetically modified cells as described herein are also provided.

In any of the compositions and methods described herein, the DNA binding domain may be part of a library of DNA binding domain-encoding nucleic acids. When the libraries are introduced into the host cell and the DNA binding domain expressed, the most specific DNA binding domain(s) from a large number of candidates in the library can be readily identified. The library may include nucleases that differ from each other in one more residues of the DNA-binding domain, for example, ZFPs that differ in one more residues in one or more recognition helix regions. In some embodiments, the library may comprise a series of guide RNAs to aid in the identification of a highly specific guide. The guide RNAs in the library may differ from each other in a number of ways, e.g. overall length, nucleoside base composition etc. Furthermore, any of the methods and compositions described herein, the DNA binding domain may then be used such that it is comprised in a ZFN or ZFN pair; a homing endonuclease with an engineered DNA-binding domain and/or a fusion of a DNA-binding domain of a homing nuclease and a cleavage domain of a heterologous nuclease; a TALEN or TALEN pair; and/or a CRISPR/Cas or Ttago nuclease system.

In any of the methods and compositions described herein, levels of reporter gene activity may be measured directly, for example by directly assaying the levels of the reporter gene product (e.g., GFP fluorescence). Alternatively, levels of the reporter gene can be assayed by measuring the levels of a downstream product (e.g., enzymatic product) of the reaction that requires function of the protein encoded by the reporter gene. In addition, in any of these methods, expression of the DNA binding protein(s) may be driven by a constitutive or inducible promoter. Furthermore, in any of the methods described herein, the DNA binding protein(s) (e.g., ZFP, or ZFN, ZFN pair, TALEN, engineered homing endonuclease and/or fusion or a naturally occurring or engineered homing endonuclease DNA-binding domain and heterologous cleavage domain, any of which may comprise the identified DNA binding domain) may be known to recognize the target sequence, for example from results obtained from another in vitro assay experiment.

In any of the compositions (e.g., reporter constructs) and methods described herein, the first and second target sites may each comprise between 12 and 100 base pairs (or any number therebetween). In certain embodiments, each target site comprises 12 to 60 base pairs (or any number therebetween), for example a paired target site that includes two target sites of 12, 13, 14, 15, 16, 17, 18, 19, 20, 21, 22, 23, 24, 25, 26, 27, 28, 29 or 30 base pairs for a total of 24 to 60 base pairs. The target sites may be contiguous or may include “skipped” bases not bound by the DNA-binding domain. Furthermore, the first and second target sites may differ at 1, 2, 3, 4, 5, 6, 7, 8, 9, 10 or more nucleotides. In certain embodiments, the first and second target sites differ in 1, 2, 3, 4, 5, 6 or 7 nucleotides. Thus, depending on the length of the target site, the homology (similarity) between the target sites may be at least 50% similar (identical), including any value between 50% and 100%, such as at least 60% homologous, at least 70% homologous, at least 75%, 80% homologous, at least 90% homologous, at least 95% homologous or at least 99% homologous to each other.

In any of the methods described herein, the DNA binding domains can be fused to and/or used with a nuclease domain to create an engineered nuclease, for example engineered meganucleases, zinc finger nucleases (ZFNs), TALE-nucleases (TALENs including fusions of TALE effectors domains with nuclease domains from restriction endonucleases and/or from meganucleases (such as mega TALEs and compact TALENs)), Ttago system and/or CRISPR/Cas nuclease systems that are used to cleave DNA at any endogenous locus. In some embodiments, the endogenous locus is related to a disease or condition such that cleavage of the endogenous gene (e.g. PD-1, Bcl11A, Htt, etc.) may be used to prevent or treat a disease or condition. In other aspects, the locus is a ‘safe harbor’ gene locus (e.g. CCR5, AAVS1, HPRT, Rosa or albumin) in the cell into which the gene is inserted. Targeted insertion of a donor transgene may be via homology directed repair (HDR) or non-homology repair mechanisms (e.g., NHEJ donor capture). The nuclease can induce a double-stranded (DSB) or single-stranded break (nick) in the target DNA. In some embodiments, two nickases are used to create a DSB by introducing two nicks. In some cases, the nickase is a ZFN, while in others, the nickase is a TALEN; a CRISPR/Cas nickase or a Cas-FokI fusion protein for use in a CRISPR/Cas system. The nuclease (e.g., ZFNs, CRISPR/Cas systems, Ttago and/or TALENs) may be provided as a polynucleotide and/or protein. The polynucleotide may be, for example, mRNA. In some aspects, the mRNA may be chemically modified (See e.g. Kormann et al, (2011) Nature Biotechnology 29(2):154-157). The polynucleotides may be provided within an expression vector comprising a polynucleotide (e.g., a plasmid, DNA minicircle, or a viral vector), encoding one or more nucleases (e.g., ZFNs, CRISPR/Cas systems, Ttago and/or TALENs) as described herein, operably linked to a promoter. A kit, comprising the compositions (e.g., selection systems, reporters, cells, transgene donors, and optionally ZFPs, CRISPR/Cas system and/or TALENs) of the invention, is also provided. The kit may comprise instructions for performing the methods of the invention, and the like.

These and other aspects will be readily apparent to the skilled artisan in light of disclosure as a whole.

### BRIEF DESCRIPTION OF THE FIGURES

FIGS. 1A to 1D depict how multiple reporters allow the simultaneous investigation of independent interactions by bacterial hybrid assay. FIG. 1A is a schematic showing the current omega-based B1H system expressed two selectable reporters (HIS3 and URA3) from the same weak promoter. Therefore, in this configuration, to test whether a protein can bind Site 1 with HIS3 (top) and/or Site 2 with URA3 (bottom) would require iterative experiments and introduce error. FIG. 1B shows novel rearrangement of the B1H reporter vector to express the selectable HIS3 and URA3 markers from separate promoters allows the assay of two independent interactions simultaneously. The addition of fluorescent markers mCherry and GFP provides a secondary, graded measure of activity for each interaction. FIG. 1C depicts how survival is dependent on the affinity of the protein DNA interaction and the selection conditions that impact each reporter independently. Here, Zif268 is expressed as an omega fusion. Zif268's consensus binding site (labeled “a. Cons”, SEQ ID NO:1) is placed in front of the URA3 reporter and paired with one of a set of binding sites of known affinity in front of HIS3 (numbered from 1 to 6 by the noted “X” fold decrease in affinity offered by each site. As a control, a sequence Zif268 will not bind to (labeled “b. Neg”, SEQ ID NO:2) is placed in front of URA3 and paired with the consensus in front of HIS3. Also shown are highly homologous sites, with changes shown in underlining as compared to consensus tested at site 1: 2× (SEQ ID NO:3); 5× (SEQ ID NO:4); 10× (SEQ ID NO:5) and 20× (SEQ ID NO:6). Log phase cells were titered in 10-fold dilutions from top to bottom on rich media or selective plates that contain 6-azauracil and either a Low (2 mM), Medium (5 mM) or High (20 mM) level of 3-AT. Survival is dependent on expression of URA3 (1a vs 1b) and related to the affinity of the interaction that drives HIS3 expression. FIG. 1D shows fluorescent output is related to the affinity of the protein-DNA interaction that drives its expression and unrelated to the selection conditions impacting the competing binding site and reporter. The same cells tested in FIG. 1C, as well as a complete set of the site 1 sequences paired with the negative “b” sequence, were grown in either rich media or selective media that impacts only URA3 expression. While HIS3 expression is not selected for, GFP output is related to the affinity of the interaction that drives the HIS3/GFP reporter and unrelated to the growth conditions that impact site 2 and URA3/mCherry.

FIGS. 2A to 2D depict selection of zinc fingers that can discriminate between similar targets. FIG. 2A is a schematic depicting the selection process. The desired and counter targets are placed in front of the promoters that drive HIS3 and URA3 expression, respectively. Target sites for CCR2 (SEQ ID NO:7) and CCR5 (SEQ ID NO:8) are shown. Zinc finger pools previously selected to bind each 3 bp sub-site of the desired target are used as PCR templates to assemble a 4-fingered library, illustrated as rainbow-colored ovals. This 4-fingered library is expressed as an omega fusion. To select 4-fingered members of this library that are able to discriminate between the desired targets, cells are grown under conditions that are inhibited by URA3 expression but required HIS3 expression. A workflow of the procedure is shown below. FIGS. 2B and 2C are graphs and FACS analysis showing that selection conditions influence the enriched amino acids that correspond to the target mismatch. Using the library described in FIG. 2A, selection for HIS3 activations but against URA3 activation increases the fraction of the population in the GFP positive, FIG. 2B shows that mCherry negative quadrant 4 in comparison to a HIS3 positive selection alone. The percent of His positive cells is shown in the left bar and the percent of His positive, URA negative cells is shown in the right bar of each indicated condition. Using the same library, FIG. 2C shows that selection for both HIS3 and URA3 activations increases the fraction of the population in the GFP positive, mCherry positive quadrant 2 in comparison to a HIS3 positive selection alone. The percent of His positive cells is shown in the left bar and the percent of His positive, URA positive cells is shown in the right bar of each indicated condition. Sequencing the zinc fingers recovered from stringent populations of these selection conditions reveal a stark difference in the amino acids enriched in the helix that corresponds to the difference in the desired and counter target. FIG. 2D shows confirmation of the binding attributes for zinc finger candidates that represent the recovered pools (p3 and p13) shown in FIGS. 2B and 2C. Zinc finger candidates are paired with the CCR5 vs CCR2 reporter vector and grown without selection. The GFP vs mCherry attributes are complementary to the selection conditions from which the candidate protein was derived. The zinc finger used are shown (N to C) above the dotplots (see, Table A).

FIGS. 3A to 3C depict MR-B1H produced zinc fingers that provide high CCR5 activity with strong discrimination against CCR2. FIG. 3A shows the CCR5 (SEQ ID NO:9) and CCR2 (SEQ ID NO:10) sequences with the 12 base pair zinc finger targets bold and underlined. Sequences are shown 5′ to 3′, but because ZFN monomers bind opposite strands of DNA, the left ZFN monomer targets the reverse complement of the sequence shown in FIG. 3A. Mismatches with the CCR2 sequence are boxed. FIG. 3B shows SELEX results for candidate zinc fingers (Table A). Candidates ID#s are listed above each SELEX plot. Target sites (SEQ IDs NO:11 and 12) are shown below the plots in bold. For the left candidates the F2 recognition helix was used to evaluate discrimination of the candidates as between GAT and GAC target subsite. For the right candidates, the F1 recognition helix was used to evaluate discrimination of the candidates as between AAG and AAA target subsites. The discriminatory base is boxed. The recovered percentage of sequences that correspond to the CCR5 or CCR2 base at this critical position are listed to the right of each plot. Those that tolerate binding to the selected-against base are shown in boxes. FIG. 3C shows the percentage of indels (insertions and/or deletions following cleavage) at CCR5 and CCR2 measured (left and middle tables) following introduction of the indicated candidate ZFN pairs in vivo. ZFNs 8266-20505 (see, U.S. Pat. No. 7,951,925) were tested in parallel for reference. The ratio of CCR5 to CCR2 indel frequency is shown in the right table. The indel frequency recovered from a control that expressed GFP rather than a nuclease pair is shown below.

FIGS. 4A to 4C depict how extending zinc finger targets eliminates off-target (CCR2) activity. FIG. 4A depicts shifting of the CCR5 target 6-nt 3′ relative to the target in FIG. 3 (bold and underlined). Mismatches with the CCR2 sequence are boxed. Each monomer of the ZFN pair is increased from 4 to 6 fingers. FIG. 4B depicts production of two overlapping 4-fingered libraries for each target. The overlapping zinc fingers in the pools of these libraries target the same sequences. Zinc fingers are selected from each pool by selection for the CCR5 sequence but against the CCR2 sequence. Enriched amino acids are shown to the right with overlapping helices boxed. These sequences were used to guide the design of 4 and 6-fingered proteins. Helices of representative candidates are listed below. See, Table A for zinc finger protein recognition helix sequences. FIG. 4C shows the percentage of indels at CCR5 and CCR2 measured (left and middle tables) following expression of the indicated ZFN pairs in vivo. Indel frequencies recovered at either target from cells that did not express a nuclease (GFP) are shown below each table. The ratio of CCR5 to CCR2 indel frequency is shown in the right table.

FIG. 5 shows a comparison of the original and shifted CCR5 targets in relation to the homologous CCR2 sequence. The top line shows the original CCR5 targets (SEQ ID NO:11) in bold and underlined and the homologous CCR2 sequence (SEQ ID NO:12) shown below. Mismatches between CCR5 and CCR2 are boxed. The aligned, shifted target is shown in the middle panel (CCR5 in SEQ ID NO:13 and CCR2 in SEQ ID NO:14). The center of this target is only 6-nt 3′ to the original target, however, by shifting and extending to an 18-nt target per monomer (similar to common TALEN architectures) we are able to pick up three CCR5:CCR2 mismatches per monomer binding site. The bottom panels shows the Left A (SEQ ID NO:15), Left B (SEQ ID NO:16), Right A (SEQ ID NO:17) and Right B (SEQ ID NO:18) target sites.

FIGS. 6A through 6D show exemplary zinc fingers generated as described herein provide high HBB activity with strong discrimination against HBD. FIG. 6A shows the HBB target (SEQ ID NO:136), mismatches to the HBD sequence are boxed. The sickle cell causing mutation that separates the left target here from the HBD sequence is denoted by the downward arrow. FIG. 6B shows the right monomer library pools (SEQ ID NOs:66-93 for left column; SEQ ID NOs:95-126 for right column) for each target (SEQ ID NO:66 and SEQ ID NO:94), including two overlapping 4-finger libraries (the overlapping zinc fingers target the same sequences). Zinc fingers are also shown in Table A and were selected from each pool by selection for the HBB sequence but against the HBD sequence. Targets are shown 3′ to 5′ to emphasize the overlap in the targets of the 4-finger selections. From each of these selections, 10 of the selected ZFPs are shown. Candidates used to design the 4 and 6-finger monomers employed as nucleases are bold and underlined. All enriched amino acids for each of the 4-finger selections are shown below as a sequence logo with the overlapping 2 fingers boxed. FIG. 6C shows exemplary left and right monomers used for testing (SEQ ID NO:130-135 for left monomers and SEQ ID NO:115, 116, 97, 105, 67, 68, 72, 76, 97 and 105 for right monomers; Table A). FIG. 6D shows the percentage of indels at HBB and HBD measured when indicated ZFN pairs were expressed in vivo. Indel frequencies recovered at either target from cells that did not express a nuclease (GFP) are shown below each table.

### DETAILED DESCRIPTION

Described herein are compositions and methods for the identification of DNA binding domains that bind to their target sites with high specificity, including DNA binding domains that exhibit minimal or no (e.g., background levels) binding activity for off-target sites. Reporter constructs comprising two or more target sites to be tested are described as are host cells comprising these reporter constructs. The reporter constructs as described herein include multiple reporters separately linked to at least two different target sites such that DNA binding activity can be independently assessed for each target site. Expression of each reporter gene is readily determined by standard techniques and the levels of reporter gene expression reflect the specificity of the nuclease for the target site.

Thus, described herein are rapid and efficient selection methods for determining the most active and most specific DNA binding domains from a panel (e.g., library) of DNA binding domains. Such selected DNA binding domains can be fused to a suitable nuclease domain (e.g. Fok1) and used to create highly active and highly specific engineered nucleases.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. No. 8,586,526.

Zinc finger and TALE binding domains can be “engineered” to bind to a predetermined nucleotide sequence, for example via engineering (altering one or more amino acids) of the recognition helix region of a naturally occurring zinc finger or TALE protein. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 6,140,081; 6,453,242; 6,534,261; and 8,586,526 see also WO 98/53058; WO 98/53059; WO 98/53060; WO 02/016536 and WO 03/016496.

A “selected” zinc finger protein or TALE is a protein not found in nature whose production results primarily from an empirical process such as phage display, interaction trap or hybrid selection. See e.g., U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970 WO 01/88197 and WO 02/099084.

“TtAgo” is a prokaryotic Argonaute protein thought to be involved in gene silencing. TtAgo is derived from the bacteria Thermus thermophilus. See, e.g., Swarts et al, ibid, G. Sheng et al., (2013) Proc. Natl. Acad. Sci. U.S.A. 111, 652). A “TtAgo system” is all the components required including, for example, guide DNAs for cleavage by a TtAgo enzyme.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted nucleases as described herein create a double-stranded break in the target sequence (e.g., cellular chromatin) at a predetermined site, and a “donor” polynucleotide, with or without homology to the nucleotide sequence in the region of the break, can be introduced into the cell. See, e.g., U.S. Pat. Nos. 7,888,121; 9,045,763; and 9,005,973. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, additional nucleases (e.g., pairs of zinc-finger nucleases or TALENs) can be used for additional single- and/or double-stranded cleavage of additional target sites within the cell.

In certain embodiments of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In any of the methods described herein, the first nucleotide sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is non-homologous to the first sequence, and is inserted into the genome by non-homologous recombination mechanisms.

Any of the methods described herein can be used for partial or complete inactivation of one or more target sequences in a cell by targeted integration of donor sequence that disrupts expression of the gene(s) of interest. Cells and cell lines with partially or completely inactivated genes are also provided.

Furthermore, the methods of targeted integration as described herein can also be used to integrate one or more exogenous sequences. The exogenous nucleic acid sequence can comprise, for example, one or more genes or cDNA molecules, or any type of coding or noncoding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Pat. Nos. 7,914,796; 8,034,598; and 8,623,618, incorporated herein by reference in their entireties.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP or TALE DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid. The term also includes systems in which a polynucleotide component (e.g., DNA-binding polynucleotide) associates with a polypeptide component to form a functional molecule (e.g., a CRISPR/Cas system in which a single guide RNA associates with a functional domain to modulate gene expression).

Expression of a fusion protein or molecule in a cell can result from delivery of the fusion protein or fusion molecule components to the cell or by delivery of a polynucleotide encoding the fusion protein to a cell or polynucleotide component of the fusion molecule, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous. In addition, the term includes molecules that associate with each other, such as CRISPR/Cas transcription factor and nuclease systems made up of polynucleotide and polypeptide components in which the components associate to form a functional transcription factor or nuclease.

With respect to fusion molecules, the term “operatively linked” can refer to the fact that each of the components performs the same function in linkage to the other component as it would if it were not so linked. For example, with respect to a fusion molecule in which a DNA-binding domain is fused to an activation domain, the DNA-binding domain and the activation domain are in operative linkage if, in the fusion molecule, the DNA-binding domain portion is able to bind its target site and/or its binding site, while the activation domain is able to upregulate gene expression. When a fusion polypeptide in which a DNA-binding domain is fused to (or associated with) a cleavage domain, the DNA-binding domain and the cleavage domain are in operative linkage if, in the fusion polypeptide, the DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “safe harbor” locus is a locus within the genome wherein a gene may be inserted without any deleterious effects on the host cell. Most beneficial is a safe harbor locus in which expression of the inserted gene sequence is not perturbed by any read-through expression from neighboring genes. Non-limiting examples of safe harbor loci that are targeted by nuclease(s) include CCR5, CCR5, HPRT, AAVS1, Rosa and albumin. See, e.g., U.S. Pat. Nos. 7,951,925 and 8,110,379; U.S. Publication Nos. 20080159996; 201000218264; 20120017290; 20110265198; 20130137104; 20130122591; 20130177983 and 20130177960 and U.S. application Ser. Nos. 14/278,903 and 14/565,014).

### Reporter Constructs

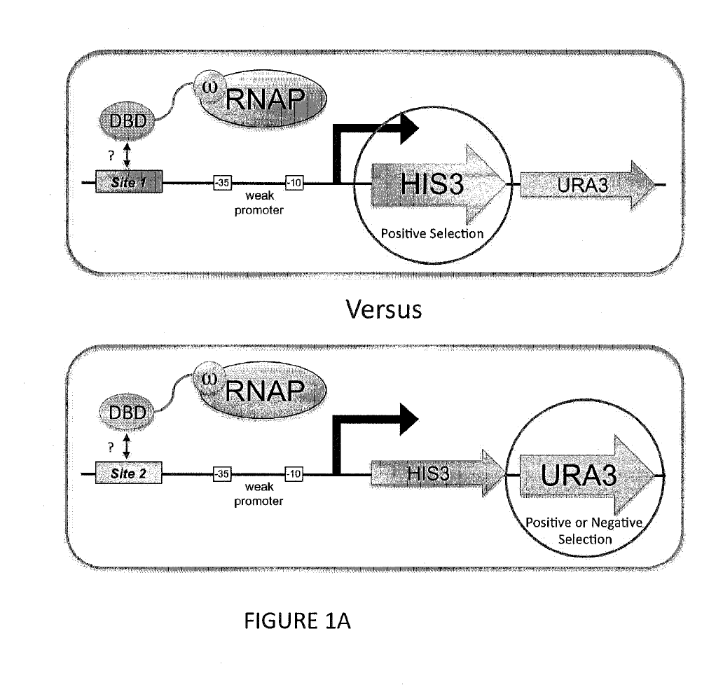

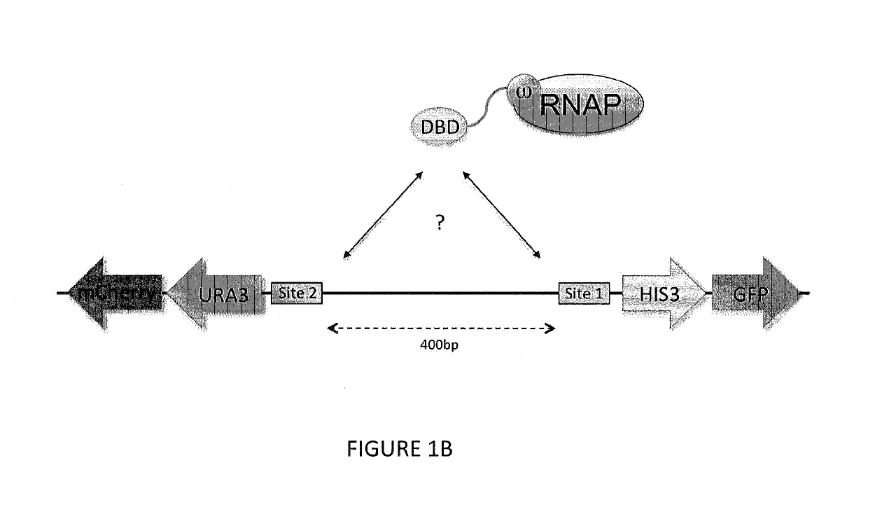

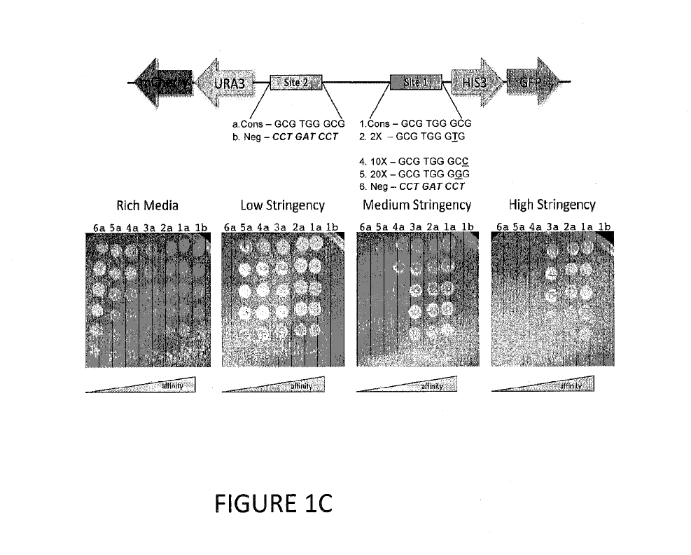

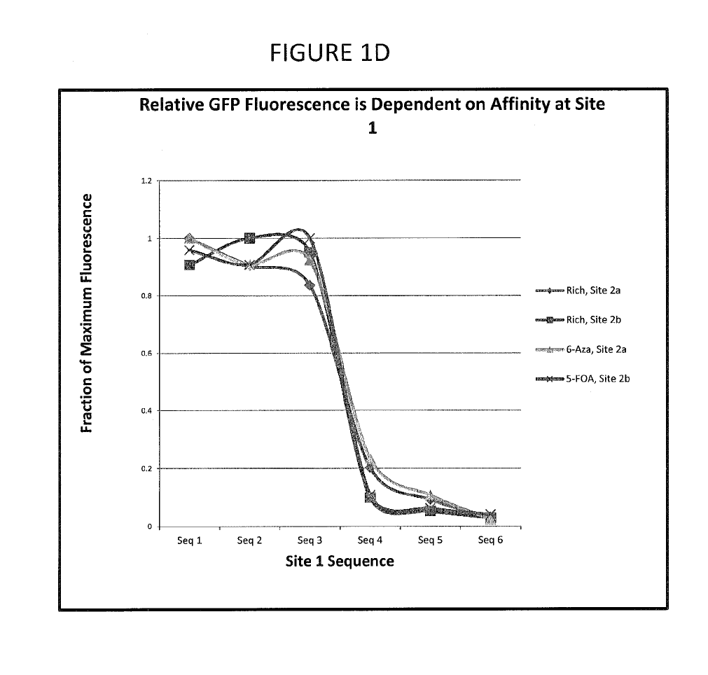

The methods and systems described herein make use of a reporter constructs comprising a sequence including two or more target sequences (sites) (e.g., two or more paired sites) for the DNA binding domains to be tested. Each target site is linked to at least one reporter gene and the expression of each reporter gene is driven by separate promoters. This reporter construct with multiple, independently-expressed reporters allows assay of DNA binding activity at the two different target sites independently and simultaneously. See, e.g., FIG. 1B. Additional reporters (e.g., fluorescent markers such as mCherry and GFP) can also be incorporated into the reporter constructs for one or more of target sites.

Thus, described herein is a reporter construct for identifying highly specific DNA binding domains, for example DNA binding domains that discriminate between two or more similar (homologous) target sites. The reporter construct comprises at least two different target sites, for example two similar paired target sites. At least one reporter gene is linked to each of the target sites and expression of the reporter genes is driven by separate promoters. In this way, binding of the DNA binding domain with respect to the different target sites can be assayed independently. One or more additional reporters may be included for one or more of the target sites, for example at least two different reporters for one or more of the target sites. The promoters for each of the multiple reporters may be the same or may be different. In addition, the promoters may be constitutive, inducible, strong or weak. The reporter genes may encode selectable markers, for example His3 and/or Ura3 or detectable reporters such as one or more fluorescent proteins (e.g., green or red).

Expression of the at least two different reporters in a host cell results in a signal that is measurable by suitable assays (and/or selection), for example by colorimetric or enzymatic assays performed on intact or lysed cells. In certain embodiments, activity of the reporter gene is determined by assaying levels of a secreted protein (e.g., the product of the reporter gene itself or a product produced directly or indirectly by an active reporter gene product). In certain embodiments, the reporter construct comprises a construct as shown in FIG. 1B.

The target sites of the reporter constructs may be of any length and may be single target site or a paired target site comprising two individual target sites recognized one member of a pair of nucleases, each nuclease comprising a DNA binding domain. In any embodiments, each target site is between about 12 and 100 base pairs (or any number therebetween) in length. In certain embodiments, each target site comprises 12 to 60 base pairs (or any number therebetween), for example a paired target site that includes two component target sites of 12, 13, 14, 15, 16, 17, 18, 19, 20, 21, 22, 23, 24, 25, 26, 27, 28, 29 or 30 base pairs each for a total of 24 to 60 base pairs. The target sites may be contiguous or may include “skipped” bases not targeted by the DNA-binding domain.

The two or more target sites in the reporter construct are different from each other in sequence. The target sites may differ at 1, 2, 3, 4, 5, 6, 7, 8, 9, 10, 11, 12, 13, 14, 15 or more nucleotides. In certain embodiments, 1, 2, 3, 4, 5, 6, 7, 8, 9 or 10 nucleotides are different as between the multiple target sites. Thus, depending on the length of the target site, the homology (similarity) between the target sites may be at least 50% similar (identical), including any value between 50% and 100%, such as at least 60% homologous, at least 70% homologous, at least 75%, 80% homologous, at least 90% homologous, at least 95% homologous or at least 99% homologous to each other.

One or more target sites for the DNA binding site(s) to be screened can be inserted into the reporter constructs by any suitable methodology, including PCR or commercially available cloning systems such as TOPO® and/or Gateway® cloning systems. Target sites can be from prokaryotic or eukaryotic genes, for example, mammalian (e.g., human), yeast or plant cells.

Any reporter gene that provides a detectable signal can be used, including but not limited, enzymes that catalyze the production of a detectable product (e.g. proteases, nucleases, lipases, phosphatases, sugar hydrolases and esterases). Non-limiting examples of suitable reporter genes that encode enzymes include, for example, MEL1, CAT (chloramphenicol acetyl transferase; Alton and Vapnek (1979) Nature 282:864 869), luciferase, β-galactosidase, β-glucuronidase, β-lactamase, horseradish peroxidase and alkaline phosphatase (e.g., Toh, et al. (1980) Eur. J. Biochem. 182:231 238; and Hall et al. (1983) J. Mol. Appl. Gen. 2:101). Reporter genes that provide a detectable signal directly may also be employed, for example, fluorescent proteins such as, for example, GFP (green fluorescent protein) or red fluorescent protein such as mCherry. Fluorescence is detected using a variety of commercially available fluorescent detection systems, including a fluorescence-activated cell sorter (FACS) system for example.

The reporter constructs may also comprise one or more selectable markers. Positive selection markers are those polynucleotides that encode a product that enables only cells that carry and express the gene to survive and/or grow under certain conditions. For example, cells that express antibiotic resistance genes (e.g. Kan^{r} or Neo^{r}) gene are resistant to the antibiotics or their analogs (e.g. G418), while cells that do not express these resistance genes are killed in the presence of antibiotics. Other examples of positive selection markers including hygromycin resistance, Zeocin™ resistance and the like will be known to those of skill in the art (see, Golstein and McCusker (1999) Yeast 15:1541-1553). Negative selection markers are those polynucleotides that encode a produce that enables only cells that carry and express the gene to be killed under certain conditions. For example, cells that express thymidine kinase (e.g., herpes simplex virus thymidine kinase, HSV-TK) are killed when gancyclovir is added. Other negative selection markers are known to those skilled in the art.

In certain embodiments, the reporter includes one or more selectable markers such as His3 and/or URA3. Cells grown in media that lacks histidine survive only when His3 is expressed (from the reporter). Thus, HIS3 expression is analyzed in the presence of a competitive inhibitor of HIS3, 3-aminotriazole (3-AT). The more 3AT in the media, the more that HIS3 expression is required for survival. Ultimately, the strength of the interaction being assayed will determine how much RNAP is recruited to the promoter, how much HIS3 is then expressed, and finally whether the cells survive the growth conditions. A negative selection can be performed with this gene based on the specific inhibitor, 5-fluoro-orotic acid (FOA) that prevents growth of the prototrophic strains but allows growth of the ura3 mutants. Ura3-cells (arising from SSA) can be selected on media containing FOA. The URA3+ cells, which contain non active ZFN, are killed because FOA is converted to the toxic compound 5-fluorouracil by the action of decarboxylase, whereas ura3− cells are resistant. The negative selection on FOA media is highly discriminating, and usually less than 10-2 FOA-resistant colonies are Ura+.

In certain embodiments, three or more reporter genes are used, for example, one or more fluorescent proteins (e.g., GFP and/or Cherry) and/or one or more positive or negative selectable markers such as HIS3 and/or URA3 (an auxotrophy marker).

### Host Cells

Also described herein is a host cell (or population of host cells) comprising any of the reporter constructs described herein. Any host cell (prokaryotic or eukaryotic) can be used. The host cell typically is a prokaryotic cell that allows for transformation of libraries of candidate DNA binding domains. In certain embodiments, the host cell is a bacterial cells such as E. coli. The reporter construct may be transiently present in the host cell. Alternatively, the reporter construct is stably integrated into the genome of the host cell.

### Nucleases

Described herein are compositions, particularly useful for creating nucleases that are useful for genomic modification. In certain embodiments, the nuclease is naturally occurring. In other embodiments, the nuclease is non-naturally occurring, i.e., engineered in the DNA-binding domain and/or cleavage domain. For example, the DNA-binding domain of a naturally-occurring nuclease may be altered to bind to a selected target site (e.g., a meganuclease that has been engineered to bind to site different than the cognate binding site). In other embodiments, the nuclease comprises heterologous DNA-binding and cleavage domains (e.g., zinc finger nucleases; TAL-effector domain DNA binding proteins; meganuclease DNA-binding domains with heterologous cleavage domains) and/or a CRISPR/Cas system utilizing an engineered single guide RNA.

A. DNA-Binding Domains

Any DNA-binding domain can be used in the compositions and methods disclosed herein, including but not limited to a zinc finger DNA-binding domain, a TALE DNA binding domain, the DNA-binding portion (e.g., single guide RNA) of a CRISPR/Cas nuclease, or a DNA-binding domain from a meganuclease.

In certain embodiments, the nuclease domain fused to the identified DNA binding domain is a naturally occurring or engineered (non-naturally occurring) meganuclease (homing endonuclease). Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. Engineered meganucleases are described for example in U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain. DNA-binding domains from meganucleases may also exhibit nuclease activity (e.g., cTALENs).

In other embodiments, the DNA-binding domain comprises a naturally occurring or engineered (non-naturally occurring) TAL effector DNA binding domain. See, e.g., U.S. Pat. No. 8,586,526, incorporated by reference in its entirety herein. The plant pathogenic bacteria of the genus Xanthomonas are known to cause many diseases in important crop plants. Pathogenicity of Xanthomonas depends on a conserved type III secretion (T3 S) system which injects more than 25 different effector proteins into the plant cell. Among these injected proteins are transcription activator-like (TAL) effectors which mimic plant transcriptional activators and manipulate the plant transcriptome (see Kay et al (2007) Science 318:648-651). These proteins contain a DNA binding domain and a transcriptional activation domain. One of the most well characterized TAL-effectors is AvrBs3 from Xanthomonas campestgris pv. Vesicatoria (see Bonas et al (1989)Mol Gen Genet 218: 127-136 and WO2010079430). TAL-effectors contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack et al (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al (2007) Appl and Envir Micro 73(13): 4379-4384). These genes are 98.9% identical in nucleotide sequence to each other but differ by a deletion of 1,575 base pairs in the repeat domain of hpx17. However, both gene products have less than 40% sequence identity with AvrBs3 family proteins of Xanthomonas. See, e.g., U.S. Pat. No. 8,586,526, incorporated by reference in its entirety herein.

Specificity of these TAL effectors depends on the sequences found in the tandem repeats. The repeated sequence comprises approximately 102 base pairs and the repeats are typically 91-100% homologous with each other (Bonas et al, ibid). Polymorphism of the repeats is usually located at positions 12 and 13 and there appears to be a one-to-one correspondence between the identity of the hypervariable diresidues (the repeat variable diresidue or RVD region) at positions 12 and 13 with the identity of the contiguous nucleotides in the TAL-effector's target sequence (see Moscou and Bogdanove (2009) Science 326:1501 and Boch et al (2009) Science 326:1509-1512). Experimentally, the natural code for DNA recognition of these TAL-effectors has been determined such that an HD sequence at positions 12 and 13 (Repeat Variable Diresidue or RVD) leads to a binding to cytosine (C), NG binds to T, NI to A, C, G or T, NN binds to A or G, and ING binds to T. These DNA binding repeats have been assembled into proteins with new combinations and numbers of repeats, to make artificial transcription factors that are able to interact with new sequences and activate the expression of a non-endogenous reporter gene in plant cells (Boch et al, ibid). Engineered TAL proteins have been linked to a FokI cleavage half domain to yield a TAL effector domain nuclease fusion (TALEN), including TALENs with atypical RVDs. See, e.g., U.S. Pat. No. 8,586,526.

In some embodiments, the TALEN comprises an endonuclease (e.g., FokI) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel et al., (2013) Nucl Acid Res: 1-13, doi: 10.1093/nar/gkt1224).

In still further embodiments, the nuclease developed by the methods and compositions herein comprises a compact TALEN. These are single chain fusion proteins linking a TALE DNA binding domain to a TevI nuclease domain. The fusion protein can act as either a nickase localized by the TALE region, or can create a double strand break, depending upon where the TALE DNA binding domain is located with respect to the TevI nuclease domain (see Beurdeley et al (2013) Nat Comm: 1-8 DOI: 10.1038/ncomms2782). In addition, the nuclease domain may also exhibit DNA-binding functionality. Any TALENs may be used in combination with additional TALENs (e.g., one or more TALENs (cTALENs or FokI-TALENs) with one or more mega-TALEs.

In certain embodiments, the DNA binding domain comprises a zinc finger protein. Preferably, the zinc finger protein is non-naturally occurring in that it is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

B. Cleavage Domains

Any suitable cleavage domain can be operatively linked to the identified DNA-binding domain to form a nuclease, such as a zinc finger nuclease, a TALEN, or a CRISPR/Cas nuclease system. See, e.g., U.S. Pat. Nos. 7,951,925; 8,110,379 and 8,586,526; U.S. Publication Nos. 20080159996; 201000218264; 20120017290; 20110265198; 20130137104; 20130122591; 20130177983 and 20130177960 and U.S. application Ser. Nos. 14/278,903 and 14/565,014.

As noted above, the cleavage domain may be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a zinc finger binding domain and two Fok I cleavage half-domains can also be used. Parameters for targeted cleavage and targeted sequence alteration using zinc finger-Fok I fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Publication WO 07/014275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Patent Publication Nos. 20050064474; 20060188987; 20070305346 and 20080131962, the disclosures of all of which are incorporated by reference in their entireties herein. Amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains.

Exemplary engineered cleavage half-domains of FokI that form obligate heterodimers include a pair in which a first cleavage half-domain includes mutations at amino acid residues at positions 490 and 538 of FokI and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. See, e.g., U.S. Pat. Nos. 7,914,796 and 8,034,598, the disclosures of which are incorporated by reference in their entireties for all purposes. In certain embodiments, the engineered cleavage half-domain comprises mutations at positions 486, 499 and 496 (numbered relative to wild-type FokI), for instance mutations that replace the wild type Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). (See U.S. Pat. No. 8,623,618). In other embodiments, the engineered cleavage half domain comprises the “Sharkey” and/or “Sharkey′” mutations (see Guo et al, (2010) J. Mol. Biol. 400(1):96-107).

Engineered cleavage half-domains described herein can be prepared using any suitable method, for example, by site-directed mutagenesis of wild-type cleavage half-domains (Fok I) as described in U.S. Pat. Nos. 7,888,121; 7,914,796; 8,034,598 and 8,623,618.

Alternatively, nucleases may be assembled in vivo at the nucleic acid target site using so-called “split-enzyme” technology (see, e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in WO 2009/042163 and 20090068164. Nuclease expression constructs can be readily designed using methods known in the art. See, e.g., United States Patent Publications 20030232410; 20050208489; 20050026157; 20050064474; 20060188987; 20060063231; and International Publication WO 07/014275. Expression of the nuclease may be under the control of a constitutive promoter or an inducible promoter, for example the galactokinase promoter which is activated (de-repressed) in the presence of raffinose and/or galactose and repressed in presence of glucose.

In certain embodiments, the nuclease comprises a CRISPR/Cas system. The CRISPR (clustered regularly interspaced short palindromic repeats) locus, which encodes RNA components of the system, and the cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cm’ proteins are involved with the natural function of the CRISPR/Cas system and serve roles in functions such as insertion of the alien DNA etc. Thus, the methods and compositions of the invention can be used to identify the most specific and active guide RNA. The reporter system can be used with a nuclease defective Cas protein in the presence of a library of guide RNA sequences. The guide RNA that in the nuclease defective Cas complex that gives the most active and specific signal is then used with a nuclease proficient Cas to create a highly active and highly specific CRISPR/Cas system for a desired cleavage target.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

Exemplary CRISPR/Cas nuclease systems targeted to safe harbor and other genes are disclosed for example, in U.S. Publication No. 20150056705.

Thus, the nuclease comprises a DNA-binding domain in that specifically binds to a target site and a cleavage domain or cleavage half-domain.

The nuclease(s) may be in the form of a library of nucleic acids encoding a variety of nucleases. Methods of making nuclease-encoding libraries are known in the art. See, e.g., U.S. Pat. Nos. 6,503,717; 7,491,531; 7,943,553; 8,618,024 and 7,700,523. The libraries may include one or more randomized amino acid residues, typically in the DNA-binding domain (e.g., recognition helix region of a ZFP or RVD of a TALE).

### Target Sites

As described in detail above, DNA-binding domains can be engineered and selected using the methods of the invention to bind to any sequence of choice, for example in a safe-harbor locus such as albumin. An engineered DNA-binding domain can have a novel binding specificity, compared to a naturally-occurring DNA-binding domain. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties. Rational design of TAL-effector domains can also be performed. See, e.g., U.S. Pat. No. 8,586,526.

Exemplary selection methods applicable to DNA-binding domains, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227.

Selection of target sites; nucleases and methods for design and construction of fusion proteins (and polynucleotides encoding same) are known to those of skill in the art and described in detail in U.S. Patent Application Publication Nos. 20050064474 and 20060188987, incorporated by reference in their entireties herein.

In addition, as disclosed in these and other references, DNA-binding domains (e.g., multi-fingered zinc finger proteins) may be linked together using any suitable linker sequences, including for example, linkers of 5 or more amino acids. See, e.g., U.S. Pat. Nos. 6,479,626; 6,903,185; and 7,153,949 for exemplary linker sequences 6 or more amino acids in length. The proteins described herein may include any combination of suitable linkers between the individual DNA-binding domains of the protein. See, also, U.S. Pat. No. 8,586,526.

DNA-binding domains of the nucleases may be targeted to any desired site in the genome. In certain embodiments, the DNA-binding domain of the nuclease is targeted to an endogenous safe harbor locus, for example an endogenous albumin locus.

### Identification of Specific DNA Binding Domains

The host cell containing reporter constructs as described herein can be used to identify the DNA binding domains that distinguish between similar binding sites.

Identification of highly specific DNA binding domains begins with introduction of a reporter construct. See, e.g., FIG. 1. The reporter construct can be episomal or can be stably integrated. After genotyping the strain for the correct integration of the reporter, the host strain is transformed with desired DNA binding domain expression vector(s). In certain embodiments, DNA binding domain expression is inducible (e.g. galactose-inducible) so that DNA binding domain expression can be induced for a selected amount of time by changing the carbon source in the culture media. The activity of the separate reporter genes (e.g. His3, Ura3, GFP, mCherry) is determined from an aliquot of the media using a suitable assay, including selection conditions for selectable reporters (e.g., FOA for Ura3 and 3-AT for His3), colorimetric assays, and/or FACs sorting. The activity obtained for each reporter reflects quantitatively that DNA binding domain's ability to bind to the target sequence.

The methods described herein allow for identification of DNA binding domains that bind a specific target site. In certain embodiments, the methods comprise introducing one or more DNA binding domains and/or one or more DNA binding domain-expression constructs (e.g., libraries) encoding one or more nucleases or one more pair of DNA binding domains into a host cell comprising a reporter construct as described herein, the reporter construct comprising a target sequence recognized by the DNA binding domain(s); incubating the cells under conditions such that the DNA binding domain(s) are expressed; and measuring the levels of reporter gene expression in the cells, wherein increased levels of reporter gene expression are correlated with increased binding of the target sequence by the DNA binding domain.

In addition, the methods described herein allow for identification of DNA binding domains that distinguish between two or more similar target sites are provided. The methods comprise introducing a DNA binding domain and/or expression constructs encoding DNA binding domains (e.g., libraries) into a host cell comprising a reporter construct as described herein; incubating the cells under conditions such that the DNA binding domain(s) are expressed; measuring the expression levels of the separate reporter in the cells; and determining the DNA binding domain that preferentially targets to one target site (e.g., by assaying reporter gene levels associated with each target site).

Thus, the multi-reporter selection system described herein to screen DNA binding domains (e.g., DNA binding domain libraries) for combinations that can discriminate between two similar targets. The system identifies DNA binding domains that discriminate between highly homologous sequences and further allows identification of DNA binding domains that manifest strong on-target binding activity with minimal or no detectible binding activity at highly homologous off-target sequences. These DNA binding domains are selected and characterized by the methods herein are then fused to a nuclease domain to create highly active and highly specific nucleases.

### Cells

Also described herein are genetically modified cells and/or cell lines, including cells that are modified by any of the nucleases described herein (e.g., Table A). In certain embodiments, the genetically modified cell or cell line comprises an insertion and/or deletion at or near any of SEQ ID NOs:28-33, 66, 94, 127, 128, 129. The modification may be, for example, as compared to the wild-type sequence of the cell. The cell or cell lines may be heterozygous or homozygous for the modification. The modifications may comprise insertions, deletions and/or combinations thereof.

The modification is preferably at or near (including within) the nuclease(s) binding and/or cleavage site(s), for example, within 1-300 (or any value therebetween) base pairs upstream or downstream of the site(s) of cleavage, more preferably within 1-100 base pairs (or any value therebetween) of either side of the binding and/or cleavage site(s), even more preferably within 1 to 50 base pairs (or any value therebetween) on either side of the binding and/or cleavage site(s). In certain embodiments, the modification is at or near a nuclease binding site shown in any of the first column of Table A. The modification may also include modifications of one or more nucleotides within the binding and/or cleavage sites.

Any cell or cell line may be modified, for example a stem cell, for example an embryonic stem cell, an induced pluripotent stem cell, a hematopoietic stem cell, a neuronal stem cell and a mesenchymal stem cell. Other non-limiting examples of cells as described herein include T-cells (e.g., CD4+, CD3+, CD8+, etc.); dendritic cells; B-cells. A descendent of a stem cell, including a partially or fully differentiated cell, is also provided (e.g., a RBC or RBC precursor cell). Non-limiting examples other cell lines including a modified beta globin sequence include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NS0, SP2/0-Ag14, HeLa, HEK293 (e.g., HEK293-F, HEK293-H, HEK293-T), and perC6 cells as well as insect cells such as Spodopterafugiperda (Sf), or fungal cells such as Saccharomyces, Pichia and Schizosaccharomyces.

The cells as described herein are useful in treating and/or preventing a disorder, for example, by ex vivo therapies. The nuclease-modified cells can be expanded and then reintroduced into the patient using standard techniques. See, e.g., Tebas et al (2014) New Eng J Med 370(10):901. In the case of stem cells, after infusion into the subject, in vivo differentiation of these precursors into cells expressing the functional transgene also occurs. Pharmaceutical compositions comprising the cells as described herein are also provided. In addition, the cells may be cryopreserved prior to administration to a patient.

Any of the modified cells or cell lines disclosed herein may show increased expression of gamma globin. Compositions such as pharmaceutical compositions comprising the genetically modified cells as described herein are also provided

### Donors

In certain embodiments, the present disclosure relates to nuclease-mediated targeted integration of an exogenous sequence into the genome of a cell using the globin gene-binding molecules described herein. As noted above, insertion of an exogenous sequence (also called a “donor sequence” or “donor” or “transgene”), for example for deletion of a specified region and/or correction of a mutant gene or for increased expression of a wild-type gene is also contemplated. It will be readily apparent that the donor sequence is typically not identical to the genomic sequence where it is placed. A donor sequence can contain a non-homologous sequence flanked by two regions of homology to allow for efficient HDR at the location of interest or can be integrated via non-homology directed repair mechanisms. Additionally, donor sequences can comprise a vector molecule containing sequences that are not homologous to the region of interest in cellular chromatin. A donor molecule can contain several, discontinuous regions of homology to cellular chromatin, and, for example, lead to a deletion of region (or a fragment thereof) when used as a substrate for repair of a DBS induced by one of the nucleases described here. Further, for targeted insertion of sequences not normally present in a region of interest, said sequences can be present in a donor nucleic acid molecule and flanked by regions of homology to sequence in the region of interest.

Polynucleotides for insertion can also be referred to as “exogenous” polynucleotides, “donor” polynucleotides or molecules or “transgenes.” The donor polynucleotide can be DNA or RNA, single-stranded and/or double-stranded and can be introduced into a cell in linear or circular form. See, e.g., U.S. Patent Publication Nos. 20100047805 and 20110207221. The donor sequence(s) are preferably contained within a DNA MC, which may be introduced into the cell in circular or linear form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. If introduced in double-stranded form, the donor may include one or more nuclease target sites, for example, nuclease target sites flanking the transgene to be integrated into the cell's genome. See, e.g., U.S. Patent Publication No. 20130326645.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome, nanoparticle or poloxamer, or can be delivered by viruses (e.g., adenovirus, AAV, herpesvirus, retrovirus, lentivirus and integrase defective lentivirus (IDLV)).

In certain embodiments, the double-stranded donor includes sequences (e.g., coding sequences, also referred to as transgenes) greater than 1 kb in length, for example between 2 and 200 kb, between 2 and 10 kb (or any value therebetween). The double-stranded donor also includes at least one nuclease target site, for example. In certain embodiments, the donor includes at least 2 target sites, for example for a pair of ZFNs or TALENs. Typically, the nuclease target sites are outside the transgene sequences, for example, 5′ and/or 3′ to the transgene sequences, for cleavage of the transgene. The nuclease cleavage site(s) may be for any nuclease(s). In certain embodiments, the nuclease target site(s) contained in the double-stranded donor are for the same nuclease(s) used to cleave the endogenous target into which the cleaved donor is integrated via homology-independent methods.

The donor is generally inserted so that its expression is driven by the endogenous promoter at the integration site, namely the promoter that drives expression of the endogenous gene into which the donor is inserted (e.g., globin, AAVS1, etc.). However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. In other embodiments, the transgene (e.g., with or without globin encoding sequences) is integrated into any endogenous locus, for example a safe-harbor locus. See, e.g., U.S. Patent Publications 20080299580; 20080159996 and 201000218264.

The transgenes carried on the donor sequences described herein may be isolated from plasmids, cells or other sources using standard techniques known in the art such as PCR. Donors for use can include varying types of topology, including circular supercoiled, circular relaxed, linear and the like. Alternatively, they may be chemically synthesized using standard oligonucleotide synthesis techniques. In addition, donors may be methylated or lack methylation. Donors may be in the form of bacterial or yeast artificial chromosomes (BACs or YACs).

The double-stranded donor polynucleotides described herein may include one or more non-natural bases and/or backbones. In particular, insertion of a donor molecule with methylated cytosines may be carried out using the methods described herein to achieve a state of transcriptional quiescence in a region of interest.

The exogenous (donor) polynucleotide may comprise any sequence of interest (exogenous sequence). Exemplary exogenous sequences include, but are not limited to any polypeptide coding sequence (e.g., cDNAs), promoter sequences, enhancer sequences, epitope tags, marker genes, cleavage enzyme recognition sites and various types of expression constructs. Marker genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence.

In a preferred embodiment, the exogenous sequence (transgene) comprises a polynucleotide encoding any polypeptide of which expression in the cell is desired, including, but not limited to antibodies, antigens, enzymes, receptors (cell surface or nuclear), hormones, lymphokines, cytokines, reporter polypeptides, growth factors, and functional fragments of any of the above. The coding sequences may be, for example, cDNAs.

For example, the exogenous sequence may comprise a sequence encoding a polypeptide that is lacking or non-functional in the subject having a genetic disease, including but not limited to any of the following genetic diseases: achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasiaossificans progressive, fragile X syndrome, galactosemis, Gaucher's disease, generalized gangliosidoses (e.g., GM1), hemochromatosis, the hemoglobin C mutation in the 6^{th} codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240).

Additional exemplary diseases that can be treated by targeted integration include acquired immunodeficiencies, lysosomal storage diseases (e.g., Gaucher's disease, GM1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias.

In certain embodiments, the exogenous sequences can comprise a marker gene (described above), allowing selection of cells that have undergone targeted integration, and a linked sequence encoding an additional functionality. Non-limiting examples of marker genes include GFP, drug selection marker(s) and the like.

Additional gene sequences that can be inserted may include, for example, wild-type genes to replace mutated sequences. For example, a wild-type Factor IX gene sequence may be inserted into the genome of a stem cell in which the endogenous copy of the gene is mutated. The wild-type copy may be inserted at the endogenous locus, or may alternatively be targeted to a safe harbor locus.

Construction of such expression cassettes, following the teachings of the present specification, utilizes methodologies well known in the art of molecular biology (see, for example, Ausubel or Maniatis). Before use of the expression cassette to generate a transgenic animal, the responsiveness of the expression cassette to the stress-inducer associated with selected control elements can be tested by introducing the expression cassette into a suitable cell line (e.g., primary cells, transformed cells, or immortalized cell lines).

Furthermore, although not required for expression, exogenous sequences may also transcriptional or translational regulatory sequences, for example, promoters, enhancers, insulators, internal ribosome entry sites, sequences encoding 2A peptides and/or polyadenylation signals. Further, the control elements of the genes of interest can be operably linked to reporter genes to create chimeric genes (e.g., reporter expression cassettes).

Targeted insertion of non-coding nucleic acid sequence may also be achieved. Sequences encoding antisense RNAs, RNAi, shRNAs and micro RNAs (miRNAs) may also be used for targeted insertions.

In additional embodiments, the donor nucleic acid may comprise non-coding sequences that are specific target sites for additional nuclease designs. Subsequently, additional nucleases may be expressed in cells such that the original donor molecule is cleaved and modified by insertion of another donor molecule of interest. In this way, reiterative integrations of donor molecules may be generated allowing for trait stacking at a particular locus of interest or at a safe harbor locus.

The following Examples relate to exemplary embodiments of the present disclosure in which the DNA binding domain comprises a zinc finger protein (ZFP). It will be appreciated that this is for purposes of exemplification only and that other proteins can be assessed and/or selected using the methods and compositions as described herein, for instance, TALEs, homing endonucleases (meganucleases) with engineered DNA-binding domains and/or fusions of naturally occurring of engineered homing endonucleases (meganucleases) DNA-binding domains and heterologous cleavage domains, and/or a CRISPR/Cas system comprising an engineered single guide RNA selected by the methods described herein.

### Examples

### Example 1: Methods

### Isolated Bacterial One-Hybrid (B1H) Activity Assay

Cells with desired omega-zinc finger & multi-reporter plasmid combinations were cultured until “cloudy” (OD600 0.1 and above but not saturated) with rotation at 37° C. Cells were pelleted and re-suspended in non-selective minimal media essentially as described in Noyes et al (2008) Nucleic Acids Res. 36(8):2547-60. Cells were expanded for 1 hour at 37° C. Cells were pelleted and washed 4 times in minimal NM media lacking histidine, uracil and IPTG. Cells were re-suspended in 1 ml of this media, tittered in 10-fold dilutions on rich plates with the appropriate antibiotics, and stored at 4° C. overnight.

Based on the overnight titer results, a similar number of cells harboring various zinc finger—binding site reporter combinations were titered in 10-fold dilutions on selective plates to provide side by side comparisons. These plates were grown for 24 hours at either 30° C. (see, U.S. Pat. No. 8,772,008) or 37° C.

### Liquid B1H Assay

### Growth Rate Test

Cells with appropriate omega-zinc finger and multi-reporter plasmids were cultured overnight in supplemented minimal media to saturation with appropriate antibiotics but no selection or counter selection pressure. Cells were diluted 1:150 into a optically clear flat bottom 96 well plate (Corning) with 150 μL of minimal media (NM), appropriate antibiotics, 3-aminotriazole, and either no URA3 inhibitor, 6-azauracil (2 pg/ml) or 5-fluoroorotic acid (2 mM) (ThermoScientific) per well.

These wells represented the no URA3 selection, positive URA3 selection, and negative URA3 selection conditions, respectively. The plate was sealed with optically clear breathable film (Sigma, Z380059) and placed in a plate reader. The plates were grown shaking at either 30° C. or 37° C., double orbital (120 rpm). OD600 measurements for each well were recorded every 10 min for 24 hours and normalized to a blank. Doubling times in log phase were calculated.

### Fluorescence Test

Cells with appropriate omega-zinc finger and multi-reporter plasmids were cultured until “cloudy” (OD600 0.1 and above but not saturated). Cells were pelleted and resuspended in non-selective minimal media (NM). Cells were expanded for 1 hour at 37° C. Cells were pelleted and washed 4 times in minimal media that lacks histidine, uracil and IPTG. Cells were resuspended in 1 ml of this media, tittered in 10-fold dilutions on rich plates with the appropriate antibiotics, and stored at 4° C. overnight.

Based on the overnight titer results, a volume of the culture held at 4° C. that contained 10 million cells was used to start a 15 ml culture of minimal media containing 100 uM IPTG and various inhibitors as indicated (6-aza (2 pg/ml), 5-FOA (2 mM), and/or 3-AT (5 mM)). These cultures were grown from 16-24 hours but not allowed to reach OD600 above 0.8. Cells were recovered and resuspended in PBS plus 0.1% Tween. The mean fluorescence of each sample was measured with a BD LSRII Multi-Laser Analyzer with HTS (BD Biosciences, Sparks, Md., USA). Mean fluorescence values were determined from at least 20,000 cells. Each zinc finger-reporter pair was assayed in triplicate.

### Pool Assembly of Zinc Finger Libraries

In principle our four-fingered zinc finger libraries were assembled as described in U.S. Pat. Nos. 6,503,717; 7,491,531; 7,943,553; 8,618,024; 7,700,523; 7,030,0215; and 7,585,849. We used our previously selected pools of individual zinc fingers as PCR templates to build four-fingered libraries guided by the desired 12-nt target. Therefore, for each 12-nt target a new four-fingered “pool library” was assembled. To create this library, individual zinc finger pools corresponding to each 3-nt subsite of the 12-nt target were used as the templates for PCR. For example, for the original right CCR5 target, 5′-AAA-CTG-CAA-AAG-3′ (SEQ ID NO:19) the AAA, CTG, CAA, and AAG pools were used as the PCR templates for each finger of the library. PCR primers were designed to provide overlap so that these PCR pools could be assembled in a second round of PCR by overlapping PCR in the order N-terminus-pool AAG-pool CAA-pool CTG-pool AAA-C-terminus (zinc fingers bind DNA anti-parallel to the 5′-3′ sequence of DNA). The 5′ and 3′-most oligonucleotides code for KpnI and XbaI restriction sites, respectively. Digestion of the final, four-fingered PCR pool assembly with these two restriction enzymes allowed cloning, in frame, into our expression vectors at the 3′ end of the omega coding sequence.

PCR reactions were carried out according to the manufacturer's guidelines using Expand High Fidelity Plus (Roche). For each individual zinc finger pool, eight, 15-20 cycle 50 ul PCR reactions were performed. The PCR products were recovered by gel purification and used as the template for the assembly rounds of PCR, again using Expand High Fidelity Plus. The final four-fingered pool assemblies were recovered by gel purification and used as the template for a final library expansion that only includes the 5′ and 3′ most oligonucleotides and 30 cycles of PCR. This final expansion was recovered by PCR purification (Qiagen) and digested with KpnI and XbaI according the manufacturer's guidelines (NEB). The digested product was recovered by gel purification (Qiagen Minelute) and eluted in a small volume of buffer (typically 10 microliters or less) to maintain high concentration. Finally, 20 to 100 μl ligations into the expression vectors were performed using T4 DNA Ligase (NEB). In each ligation digested vector is present at a concentration of 1 ug/10 μl of ligation. Digested library insert is added to a final concentration that provides a 5× insert to vector molar ratio. For most of our libraries this is approximately 500 ng of insert per 1 ug of vector. Ligations were incubated at 16° C. overnight (minimum of 16 hours). The ligation was ethanol precipitated and resuspended in 1 ul of water per 1 ug of vector backbone used in the ligation.

In particular, four fingered zinc finger pools were assembled as the following sequences, with the intention of overlapping the C-terminal 2 fingers of the “N-4” and the N-terminal 2 fingers of the “C-4” when six fingered proteins were required. 6-fingered proteins were synthesized based on the selection results. The approach also enabled synthesis to reflect human coding bias. Therefore, many of the zinc fingers used in the cellular studies were based on the selection results but synthesized with optimal coding sequences. NNS bases below represent the helices of the zinc fingers and the bases that were randomized in the original zinc finger pools.

N-4
(SEQ ID NO: 20)
GGTACCGAGCGCCCATTCCAGTGTCGAATCTGCATGCGTAACTTCAGTNN
SNNSNNSNNSCTGNNSNNSCACATCCGCACCCACACCGGCGAGAAGCCTT
TTGCCTGTGACATTTGTGGGAGGAAGTTTGCCNNSNNSNNSNNSCTGNNS
NNSCATACCAAAATCCATACAGGTTCCCAGAAACCGTTTCAATGCAGGAT
ATGCATGCGTAACTTCAGTNNSNNSNNSNNSCTGNNSNNSCACATCCGCA
CCCACACCGGCGAGAAGCCTTTTGCCTGTGACATTTGTGGGAGGAAGTTT
GCCNNSNNSNNSNNSCTGNNSNNSCATACCAAAATCCATTTACGTGGATC
CTAAGTCTAGA
C-4
(SEQ ID NO: 21)
GGTACCGAGCGCCCATTTCAATGCAGGATATGCATGCGTAACTTCAGTNN
SNNSNNSNNSCTGNNSNNSCACATCCGCACCCACACCGGCGAGAAGCCTT
TTGCCTGTGACATTTGTGGGAGGAAGTTTGCCNNSNNSNNSNNSCTGNNS
NNSCATACCAAAATCCATACCGGCAGCCAGAAGCCATTTCAGTGCCGCAT
TTGCATGCGTAACTTCAGTNNSNNSNNSNNSCTGNNSNNSCACATCCGCA
CCCACACCGGCGAGAAGCCTTTTGCCTGTGACATTTGTGGGAGGAAGTTT
GCCNNSNNSNNSNNSCTGNNSNNSCATACCAAAATCCATTTACGTGGATC
CTAAGTCTAGA

### Zinc Finger Protein Selections

To select four-finger proteins able to bind 12-nucleotide targets of interest, the library assemblies described above were paired with the appropriate multi-reporter vector and transformed into our selection strain. For each transformation 1 ul of the ligation (loosely representing 1 ug of library vector) was paired with 1 ul of multi-reporter vector (500 ng-1 ug) and transformed into our ΔrpoZ selection strain by electroporation. In this way the library build was recovered and assayed in one step. Typically, 1 ul of library gave us 5×10⁷ transformants in our selection strain prepared as previously described. Therefore, to assay well over 108 library members a standard selection would include 4 or 5 transformations. After electroporation, the cells were expanded in rich media (SOC) for 1 hour at 37° C. The cells were pelleted and resuspended in 10 ml of non-selective minimal media (NM) that contained the kanamycin and ampicillin. The cells were again expanded for 1 hour at 37° C. The cells were pelleted and washed 4 times in NM without uracil or histidine. The cells were resuspended in 1 ml of NM without uracil and histidine. 20 ul of this resuspension was titered in 10 fold dilutions on rich media plates while the remaining 980 μl stored at 4° C. overnight.

The following day, cell titers provide a cell count/volume. 2×10⁸ cells were plated on NM plates containing 5 mM 3AT to provide a low stringency positive selection and remove non-functional zinc fingers. These plates were incubated at 37° C. for 36-48 hours. In all cases reported here at least 10,000 cells survived this low stringency selection. After incubation, cells were harvested, DNA recovered and precipitated. This DNA was transformed again with the appropriate multi-reporter plasmid. Again, after electroporation the cells were expanded in rich media (SOC) for 1 hour at 37° C. The cells were pelleted and resuspended in 10 ml of non-selective minimal media (NM) that contained the kanamycin and ampicillin. The cells were again expanded for 1 hour at 37° C. The cells were pelleted and washed 4 times in NM without uracil or histidine. The cells were resuspended in 1 ml of NM without uracil and histidine. 20 ul of this resuspension was titered in 10 fold dilutions on rich media plates while the remaining 980 ul stored at 4° C. overnight. Based on the overnight titer results, a volume that contains 1×10⁷ cells was used to start a 15 ml culture of minimal media containing 100 uM IPTG and 10 mM 3AT and 2 mM 5-FOA. These cultures were grown for 24-30 hours at 30° C. but not allowed to reach OD600 above 0.8. Cells were recovered and resuspended in PBS plus 0.1% Tween for sort preparation.

### Fluorescence Activated Cell Sorting (FACS) and Recovery

Cells prepared as above were sorted directly into rich media (SOC) using a BD FACSVantage SE w/DiVa instrument (BD Biosciences, San Jose, Calif.) at 16 psi with a 70 micron nozzle using sterile PBS as sheath fluid. Cells were characterized using forward and side scatter parameters and GFP and mCherry fluorescent proteins were excited via 488 nm and 568 nm laser lines, respectively. Emitted fluorescence was collected using a 530/30 bandpass filter for GFP and a 600 longpass filter for mCherry. Data were acquired and analyzed using FACSDiVa software (BD Biosciences). 30,000 events were collected for each sorted population. A volume of the recovered events were plated on rich media to recover 250-500 colonies of bacteria and grown overnight at 37° C. From 24-48 individual colonies, the zinc finger coding sequences were amplified by PCR and sequenced for each target selection. Coding sequences were translated enriched amino acids compared for analysis.

### SELEX Studies

Experimental conditions were essentially as described (Nat Biotechnol 29, 143). Briefly, an oligonucleotide target library was synthesized bearing the sequence: 5′-CAGGGATCCATGCACTGTACGCCCNNNNNNNNNNNNNNNNNNNNNNGGGCCAC TTGACTGCGGATCCTGG (SEQ ID NO:22) where “N” denotes a mixture of all four bases. The library was converted to double-stranded duplex by annealing 2 nmol of library oligo with 6 nmol of 3′ library primer (5-CCAGGATCCGCAGTCAAGTGG, SEQ ID NO:23) in 100 μL×PCR Master (Roche) supplemented to 1.2 mM of each dNTP and 5 mM MgSO4, followed by incubation at 95° C. for 2 min, 94° C. for 5 min, 58° C. for 5 min, and 72° C. for 15 min.

For the first assay cycle, ZFNs were expressed directly from plasmid templates using a TnT coupled transcription-translation system (Promega) and the manufacturer's recommended conditions with buffers supplemented to 10 mM ZnCL2. Expressed ZFNs contained a triple Flag tag fused to their N-terminus. 12 μL of TnT reaction mix was then mixed with 200 pmol of library duplex in a total volume of 100 μL of binding buffer (50 mM DTT, 10 μM ZnCL2, 5 mM MgCl2, 0.01% BSA Fraction V, 100 mM NaCl in PBS (calcium-free)). After incubation for 50 min protein-DNA complexes were captured on anti-FLAG M2 magnetic beads (SIGMA) and washed five times with wash buffer (5 mM DTT, 10 μM ZnCL2, 5 mM MgCl2, 0.01% BSA Fraction V, 100 mM NaCl in PBS (calcium-free)). Bound target was PCR-amplified using the 3′ library primer (above) and a 5′ library primer (5′-CAGGGATCCATGCACTGTACG, SEQ ID NO:24), and the resulting amplicon was used as input for additional cycles of enrichment. Protein expression and binding conditions for these subsequent cycles were identical to the conditions used in the first round. After three cycles, recovered DNA fragments were sequenced using an Illumina MiSeq system. The protocol for adding the Illumina sequencing primers and sequencing is as described in the section for off-target analysis.

SELEX FASTQ sequences from the MiSeq were adapter trimmed using SeqPrep. SELEX library sequences were further filtered by custom python scripts for correct length and fixed flanking region composition (exact match). 200 randomly sampled filtered sequences were used as input to the GADEM motif discovery program with options maskR=0 fullscan=0 gen=3. Position frequency matrices discovered by GADEM (Li (2009) J Comput Biol 16:317) were then aligned to the intended sequence and reverse-complemented if necessary. Matrices longer than the intended sequence were trimmed to only those regions overlapping the intended sequence according to the highest-scoring alignment, yielding the final matrices provided in FIG. 1.

### Gene Modification of Endogenous CCR5, CCR2

In order to screen ZFN pairs for NHEJ-mediated gene modification, K562 cells were cultured in RPMI1640 media (Invitrogen) supplemented with 10% (v/v) FBS, 2 mM L-glutamine, 100 U/ml penicillin, and 100 mg/ml streptomycin. Cells (1-2×105) were nucleofected with expression plasmids (400 ng each) using the Amaxa 96-well shuttle system (Amaxa Biosystems/Lonza) according to manufacturers' instructions (setting 96-FF-120). Cells were collected 3 days post-transfection and genomic DNA was extracted using the QuickExtract DNA Extraction Solution (Epicentre Biotechnologies) according to suppliers' instructions. Frequency of gene modification by NHEJ was evaluated by deep sequencing using an Illumina MiSeq and the appropriate primers.

### Capture Assay

To capture IDLV at sites of ZFN cleavage, K562 cells were cultured in RPMI medium supplied with 10% Fetal Bovine Serum. One day before ZFN transfection, cells (1.5×105) were infected with IDLV at an MOI of 100. Approximately 20 hours later, cells (2×105) were nucleofected (Lonza 96-well shuttle system, Nucleofector SF Solution, and Program 96-FF-120) with each pair of ZFN expressing plasmids. Nucleofections were performed in triplicate, using 200 ng of each plasmid, for CCR5-targeted ZFNs, and in quadruplicate, using either 400 ng or 800 ng of each plasmid for HBB-targeted ZFNs. After 1 day, cultures were transferred to a 6-well plate.

On day 14 and day 28 post-transfection, genomic DNA was isolated (Qiagen DNeasy Blood & Tissue Kit) and processed to isolate insert-genome junctions essentially as described (Schmidt et al. (2007) Nature Methods 4:1051-1057; steps 1-38), except for the use of an 8 second extension time, and annealing temperatures of 53° C., 47° C., and 50° C. for each amplification step. Candidate products were then processed for high throughput sequencing via MiSeq using standard methods.

DNA sequence reads were then processed as follows: first, nonidentical reads were filtered for correct priming and adapter sequences, and the resulting sequences mapped to the genome. Next, junction coordinates were mapped and hits within 1 kb of each others were merged into clusters while keeping counts of integration events. Next, to reduce background signal from capture into random, cell cycle, or environmentally induced DSBs, clusters were filtered to contain integrations from at least 2 out of 3 replicates of ZFN treated samples and at most 1 out of 3 replicates of control were scored as potential targets. These clusters were ranked by the total number of unique integrations in the ZFN treated samples.

### Off-Target Analysis

For the off-target analysis, K562 cells were transfected with ZFN-expressing plasmids and cultured essentially as described above in the section ‘Gene modification of endogenous CCR5, CCR2’. Amplicons from candidate off-target loci were then amplified with the following optimal conditions: amplicon size of 200 nucleotides, a Tm of 60° C., primer length of 20 nucleotides, and GC content of 50%). Adapters were added for a second PCR reaction to add the Illumina library sequences (ACACGACGCTCTTCCGATCT forward primer, SEQ ID NO:25, and GACGTGTGCTCTTCCGAT reverse primer, SEQ ID NO:26), followed by MiSeq sequencing using standard methods.

Genomic DNA was purified with the Qiagen DNeasy Blood and Tissue Kit (Qiagen). Regions of interest were amplified in 50 μL using 250 ng of genomic DNA with Phusion (NEB) in Buffer GC with 200 μM dNTPs. Primers were used at a final concentration of 0.5 μM and the following cycling conditions: Initial melt of 98° C. 30 sec, followed by 30 cycles of 98° C. 10 sec, 60° C. 30 sec, 72° C. 15 sec, followed by a final extension 72° C. 10 min. PCR products were diluted 1:200 in H2O. 1 μL diluted PCR product was used in a 10 μL PCR reaction to add the Illumina library sequences with Phusion (NEB) in Buffer GC with 200 μM dNTPs. Primers were used at a final concentration of 0.5 μM and the following conditions: Initial melt of 98° C. 30 sec, followed by 12 cycles of 98° C. 10 sec, 60° C. 30 sec, 72° C. 15 sec, followed by a final extension 72° C. 10 min. PCR products were pooled and purified using the Qiagen Qiaquick PCR Purification Kit (Qiagen). Samples were quantitated with the Qubit dsDNA HS Assay Kit (Life Technologies). Samples were diluted to 2 nM and sequenced on an Illumina MiSeq Instrument (Illumina) with a 300 cycle sequencing kit.

### Example 2: Establishment of a Multi-Reporter Selection System

The omega-based bacterial one-hybrid (B1H) system has proven a simple and extremely sensitive method for the investigation of protein-DNA interactions. See, e.g., Meng and Wolfe (2006) Nature Protocols 1(1): 30-45. This system differentiates itself from other bacterial hybrid assays through the employment of the omega subunit of RNA polymerase (rpoZ) as the fusion partner to the protein of interest. In this way, omega acts as an activation domain through recruitment of the polymerase. Omega is a nonessential component of the core holoenzyme allowing selections to be carried out in an rpoZ knockout strain and therefore in the absence of competition from endogenous omega. The lack of competition allows activation of the reporter even at low levels of fusion protein expression and the recovery of interactions with a large range of affinities. As a result, the method has allowed the characterization of transcription factor DNA-binding specificities for most common DNA-binding domain families as well as the selection of synthetic homeodomains and zinc fingers with novel specificities.

To provide highly effective DNA binding proteins that could discriminate homologous sequences, we first created a B1H system in which its two selectable markers (HIS3 and URA3; FIG. 1A) are expressed from independent promoters to allow for simultaneous selection for or against activation of these two reporter genes (FIG. 1B).

To test whether our novel system accomplished this, we compared the activity of the reporters while varying known protein-DNA interactions that drive their expression. In particular, we expressed the three-finger Zif268 zinc finger protein as a direct fusion to omega. We fixed the Zif268 consensus target sequence upstream of the promoter that drives the URA3 reporter and paired this with alternative Zif268 targets exhibiting a range of different Kds upstream of the HIS3 reporter.

Bacteria bearing each Zif268-binding site combination were grown to log phase, titered, and plated on selective media. Only cells that offered a functional interaction to drive URA3 survived the presence of 6-azauracil (6-aza) while the affinity of the interaction that drives HIS3 determined survival at various stringency 3-amino triazole (3-AT) concentrations (FIG. 1C). The reverse of this was also true when the sequences in sites 1 and 2 were switched.

We confirmed these observations in follow up studies of doubling time in liquid media. omega-zif268 was expressed in combination with its consensus target sequence driving HIS3/GFP and one of a suite of binding sites driving URA3/mCherry. Cells were then grown in liquid cultures, either without selection, or with selection for HIS3 activation coupled with a negative or positive selection of URA3. These tests were done at both 30° C. and 37° C. The results demonstrated that doubling times were clearly related to the affinity of the interactions that drive URA3 and the selection conditions.

In sum, our system allows for maintaining a desired protein-DNA interaction that drives one reporter, while also allowing for the interaction that drives the secondary reporter to be selected for, or against, through the addition of inhibitors in the media.

By separating the HIS3 and URA3 reporters, we are able to investigate two independent interactions simultaneously. Still, survival alone does not allow us to differentiate between the attributes of multiple, functional interactions, only indicating that they all surpass the survival threshold required of the selection. Therefore, fluorescent reporters were also added to each of the selectable markers to provide a secondary and more graded measure of activity. A GFP cassette is expressed from the same promoter that drives HIS3 expression and mCherry expressed from the promoter that drives URA3 (FIG. 1B).

Zif268 was used to initially demonstrate the utility of this approach. While fixing the interaction that drives URA3 expression, we tested the same set of binding sites as above to drive HIS3 and therefore, GFP expression (FIG. 1D). With this system we observed that under varying conditions of URA3 selection (for, against, or neutral), mCherry expression is related to the selection conditions but is otherwise constant. We also found that, as expected, GFP expression is related to the affinity of the protein-DNA interaction that drives the HIS3/GFP promoter and unrelated to the URA3-focused experimental conditions (FIG. 1D).

These results confirm that selection conditions designed to influence URA3 expression do not impact the HIS3/GFP transcription. Therefore, activation of the reporter is a function of the protein-DNA interaction that drives its expression and the fluorescent output is a secondary measure of the activity that interaction offers.

### Example 3: Selection of Proteins by Target Discrimination

Having shown that that the MR-B1H system can function as a reporter to compare the relative strengths of two interactions, we next sought to demonstrate that this system can be used to select proteins with new binding properties. We first prepared four-fingered libraries using our recently reported, complete set of selected zinc finger pools as templates for library assembly. Briefly, zinc fingers were amplified from previously selected pools for each of the 64 possible 3-nt targets with both zif268 finger 2 and finger 3 libraries. Four-fingered libraries were constructed by alternating finger 2 and finger 3 pool amplifications that would correspond to the target of interest. Using the CCR5 right target as an example (AAA-CTG-CAA-AAG (SEQ ID NO:27), PCR primers were design to amplify each finger pool (top) in such a way as to provide overlapping linker sequences that allow assembly in the order N-terminus-AAGf2pool-CAAf3pool-CTGf2pool-AAAf3pool-C-terminus. In a second round of PCR, the AAGf2pool-CAAf3pool and CTGf2pool-AAAf3pool pairs were assembled using the designed linker overlap. In the final assembly, the middle, 6 amino acid linker overlap was used to assemble the full-length four-fingered pool library. A final round of PCR was used to expand this assembly. DNA was recovered, digested with restriction enzymes complementary to sites installed in the 5′ and 3′ extension primers (KpnI and XbaI) and ligated into the omega expression vector.

In this way we circumvented inter-finger complications that often arise when neighboring fingers of known specificity are designed next to one another. Rather, we select combinations of zinc fingers from four-fingered libraries that are most compatible with one another.

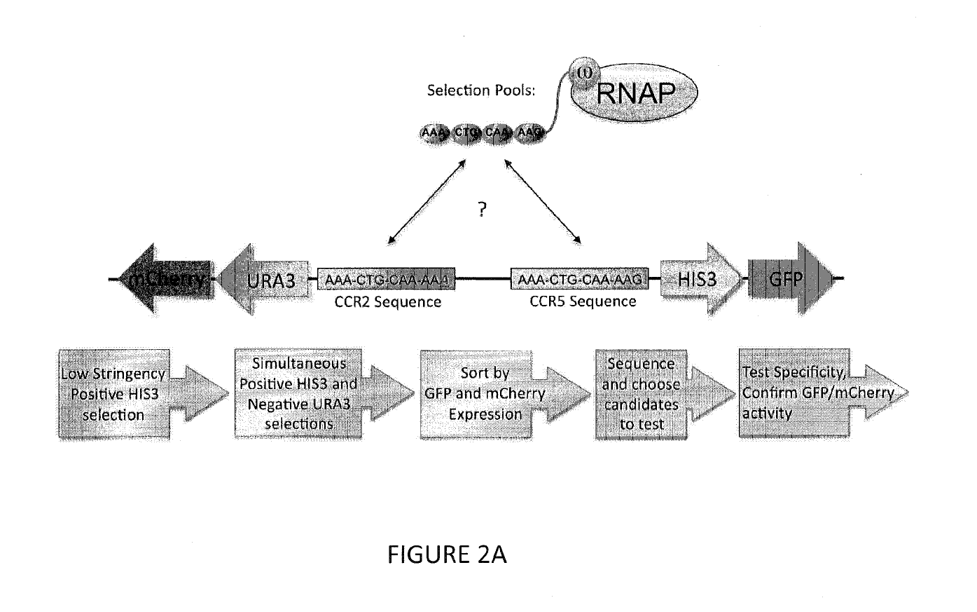

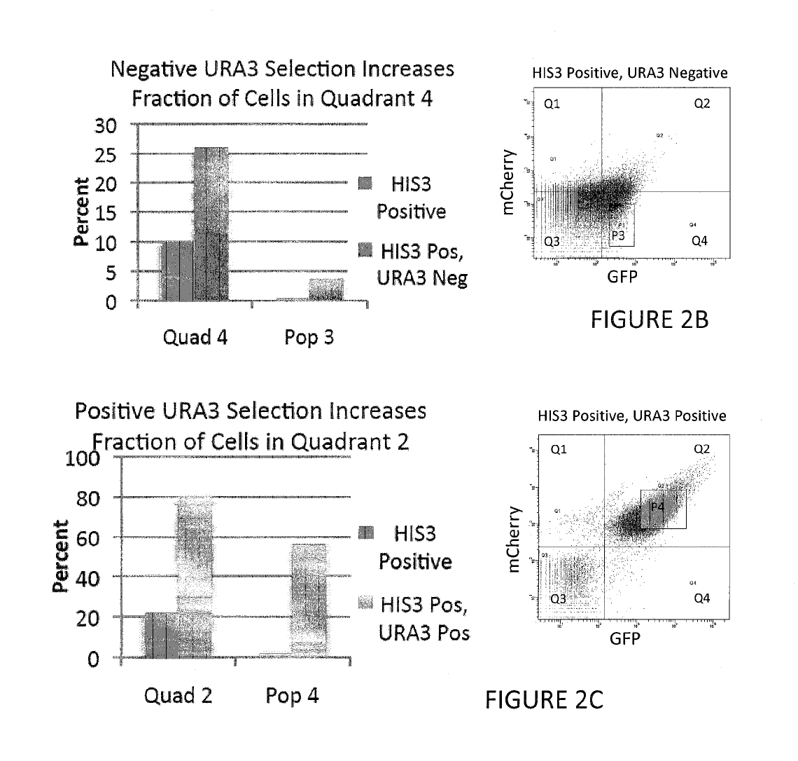

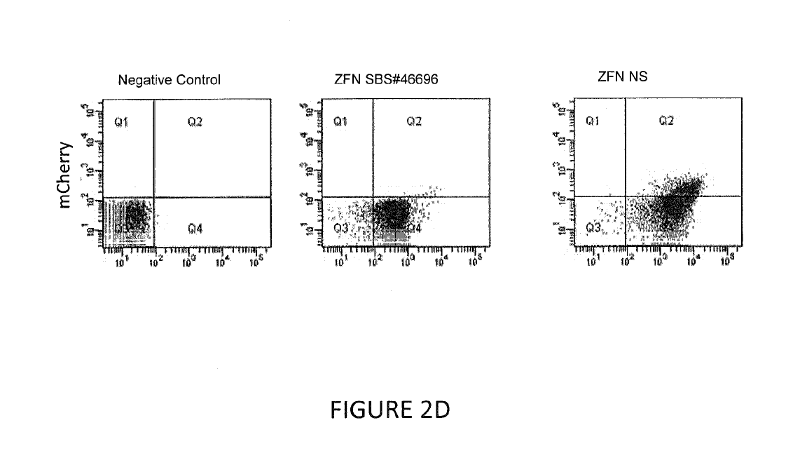

To expand the utility of the MR-B1H system, we screened our libraries, created from our individual pools, to uncover proteins able to discriminate between related sequences. For our initial study, we targeted a well-characterized sequence within the CCR5 gene, at which indels can mediate efficient functional inactivation and cellular resistance to HIV infection. This locus is targeted by a ZFN dimer currently in clinical studies. See, e.g., U.S. Pat. No. 7,951,925 and Tebas et al (2014) New Eng J Med 370(10):901. This target provided an attractive initial test of our selection system, since it exhibits substantial sequence identity with a second sequence in the human genome (within the homologous CCR2 gene), and the availability of highly active and specific published reagents for this target would provide a benchmark against which to gauge the performance of any selected ZFNs. To utilize this tool, we modified the MR-B1H system to select zinc finger arrays able to bind the CCR5 target and provide discrimination against the CCR2 target (FIG. 2A).

The 12-nt sequence targeted by the published 3′ CCR5 ZFN monomer (right target) was installed upstream of the HIS3/GFP reporter (FIG. 2A). The homologous CCR2 sequence (matching at 11/12 bases) was installed upstream of the URA3/mCherry reporter. ZFP array libraries were expressed as omega-fusions and paired with this CCR5-focused MR-B1H reporter vector.

A low stringency, HIS3 positive selection was performed to remove non-functional arrays from the library. The surviving library members were pooled, again paired with the reporters, and selected for activation of HIS3 (CCR5 target), with a secondary selection for, against, or neutral for URA3 (CCR2 target). When activation of URA3 was selected against the number of cells above the GFP background but below the mCherry threshold increased by 2.5 fold over cells grown with HIS3 selection alone (FIG. 2B, quadrant 4). Moreover, these conditions produced a stringent population of high GFP and low mCherry activity (FIG. 2B) enriched by 10-fold.

Conversely, when activation of URA3 is selected for, 80% of the enriched cells are both GFP and mCherry positive (FIG. 2C), a 4-fold enrichment over the HIS3 selection alone. Populations of these cells were recovered and the ZFPs they harbor were sequenced. The amino acids enriched in the alpha helix of the N-terminal finger, were noticeably different depending on the URA3 selection conditions. Based on the canonical model of ZFP-DNA recognition, this first helix should bind to the 3′ AAG and AAA sequences that differentiate BS1 from BS2 of the reporter, respectively.

Candidate ZFPs that represent these populations were tested again with the reporters to confirm the fluorescent activity, and thus their DNA-binding attributes, in the absence of selective pressure.

These studies confirmed that ZFPs selected from arrays from combinatorial libraries that offer fine-tuned attributes by modifying selection conditions and recovering fluorescent populations that represent the desired characteristics (e.g., distinguishing between a single base pair different in a target subsite.

### Example 4: Fine-Tuned ZFNs Offer Improved Discrimination Between CCR5 and CCR2 In Vivo

The MR-B1H system was able to provide ZFPs that function with fine-tuned specificity in E. coli, however, our goal was to provide target discrimination in human cells. To test our selected ZFPs outside of bacteria, we first repeated the procedure above to select ZFPs able to bind the published 5′ CCR5 target (left target) while discriminating against the CCR2 sequence. Briefly, as shown in FIG. 2, the CCR5 left target site LEFT target was placed in front of the promoters that drive HIS3 expression, with the corresponding CCR2 sequence in front of URA3. Zinc finger pools previously selected to bind each 3 bp sub-site of the desired target were used as PCR templates to assemble a 4-fingered library, illustrated as rainbow-colored ovals. To select 4-fingered members of this library that were able to discriminate between the desired targets, cells were grown under conditions that are inhibited by URA3 expression but required HIS3 activation.

Selection for HIS3 activations but against URA3 activation increased the fraction of the population in the GFP positive, mCherry negative (quadrant 4) cells in comparison to a HIS3 positive selection alone. Selection for both HIS3 and URA3 activations using the same library increased the fraction of the population in the GFP positive, mCherry positive quadrant 2 in comparison to a HIS3 positive selection alone. Sequencing of the zinc fingers recovered from stringent populations of these selection conditions allowed a comparison of the amino acids enriched in the helix that corresponds to the difference in the desired and counter target.

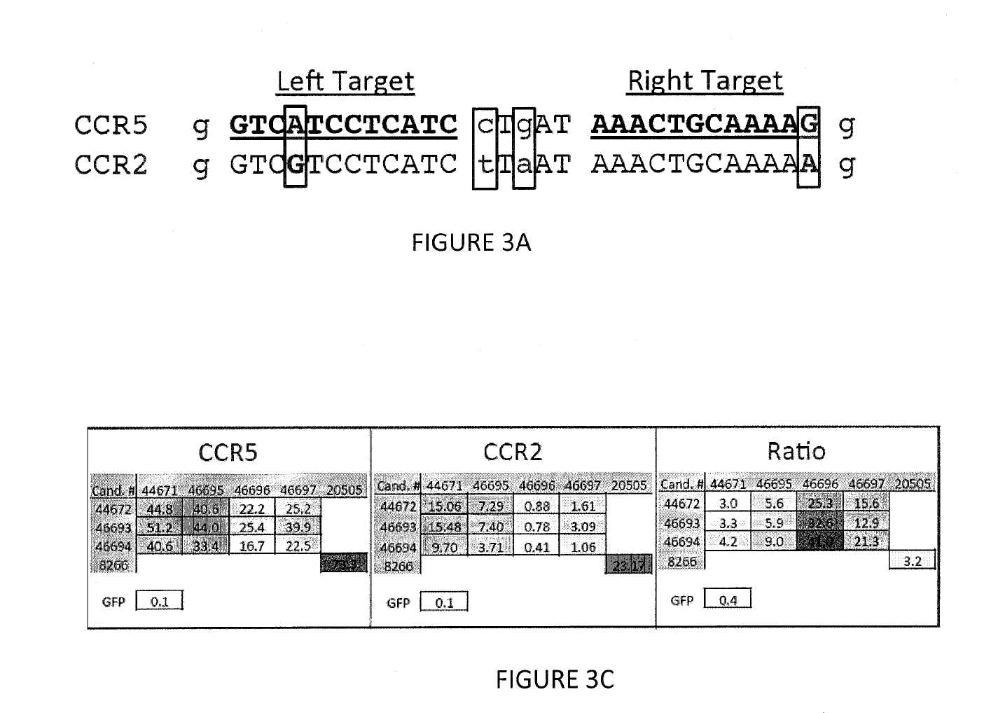

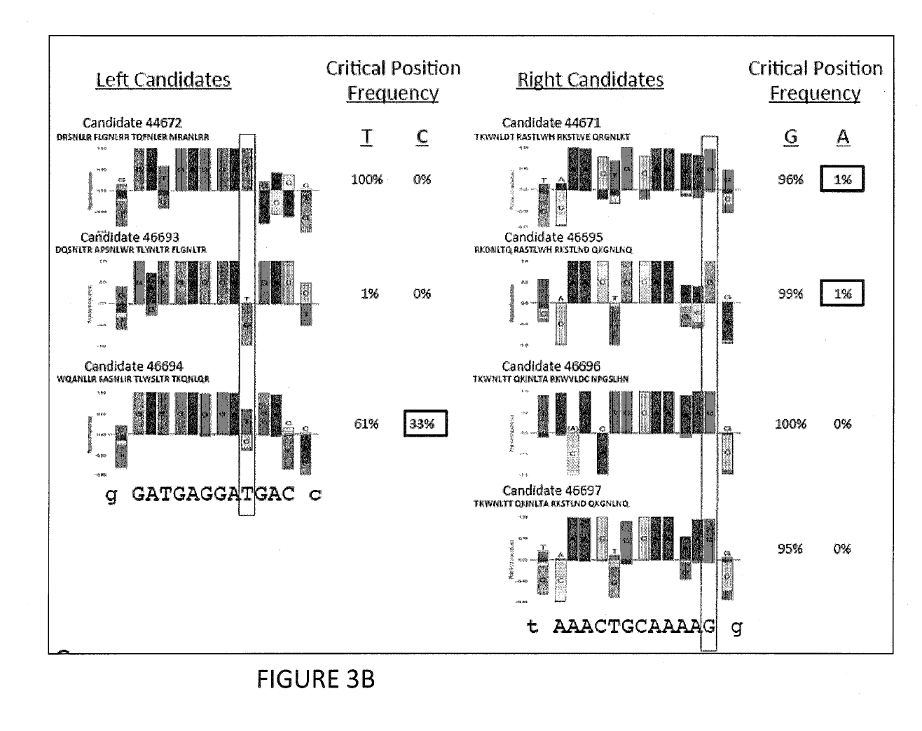

Next, we assessed the DNA-binding specificity of a set of selected “left” and “right” ZFPs using systematic evolution of ligands by exponential enrichment (SELEX). These revealed generally good specificity by the selected ZFPs, with several exhibiting a marked preference for the intended target base at those positions differing between CCR5 and CCR2 (FIGS. 3A and 3B). Next, we expressed these candidates as ZFNs in K562 cells and quantified their activity at the CCR5 and CCR2 targets (FIG. 3C). For comparison, the published set of CCR5 ZFNs were tested in parallel with the MR-B1H produced ZFNs. We found that our selected ZFNs were highly active, yielding up to 50% indels at CCR5. Moreover, many were also highly selective for CCR5 vs CCR2, with half yielding a modification ratio of >12. Interestingly, while the left monomers had some influence, the CCR2 activity of the MR-B1H produced ZFNs appeared to be primarily related to the specificity of the right monomer and its ability to bind the adenine that differentiates CCR2 from CCR5 (see SELEX results, FIG. 3B). Those with no SELEX evidence of binding adenine at this position (candidates 46696 and 46697) offer CCR5 to CCR2 indel ratios that range from 12.9 to 41. For comparison, the previously published ZFNs yielded 73% modification of CCR5 in this study, with a 3-fold preference vs cleavage of CCR2.

### Example 5: Extended ZFN Monomers Eliminate Unwanted Activity by Discriminating Against the Divergence of Homologous Targets

The MR-B1H system described herein can uncover ZFPs with strong discrimination between two targets that differ by a single base pair, even when we have restricted ourselves to duplicate an exact target in the literature. However, with a complete C2H2 ZF pool set we are not limited by sequence and have the flexibility to slightly shift the zinc finger target, if advantageous, and still remain in close proximity to the sequence to be modified. Therefore, we reasoned that by increasing the number of fingers per monomer and maximizing the counter selection by focusing on the divergence between homologous targets, we could further improve the discrimination that our ZFNs were able to offer.

### A. CCR5

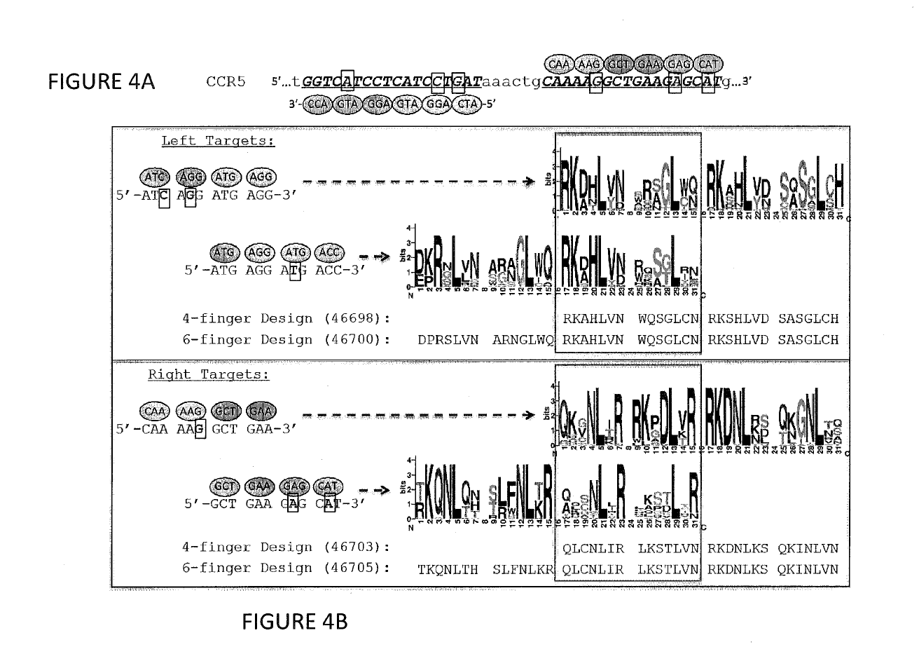

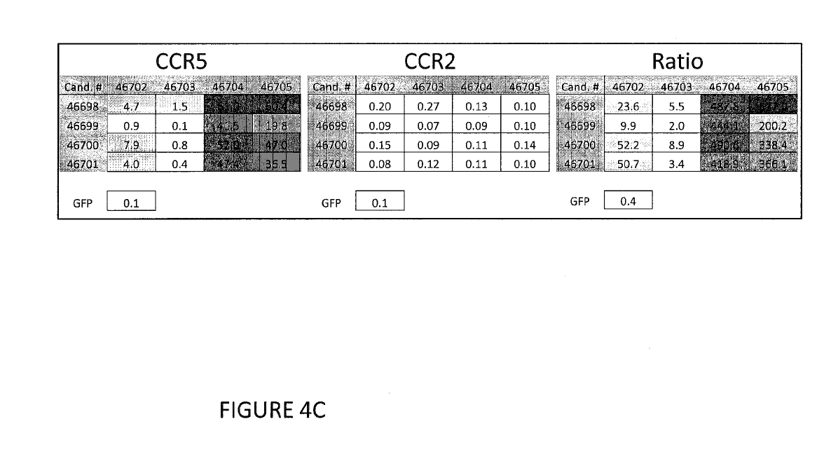

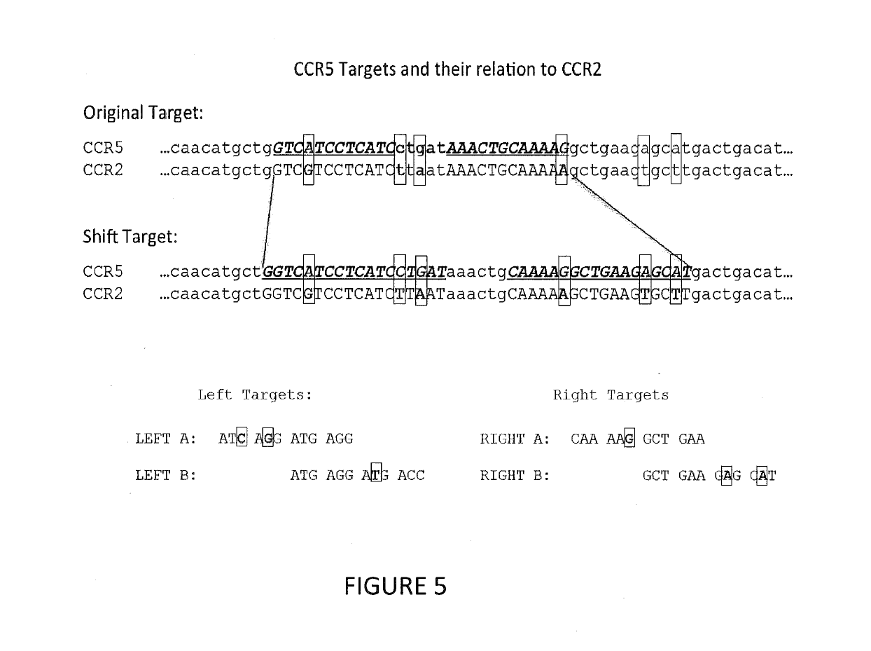

To test this approach, we shifted the CCR5 target 6 base pair 3-prime as shown in FIG. 5. We also increase the number of mismatches between the CCR5 and CCR2 by extending each ZFP monomer to contain 6 fingers per monomer (FIG. 4A). To limit library size and maintain the complexity for each finger, zinc fingers were selected from two overlapping 4-fingered libraries to bind these targets (FIG. 4B). In this way, the 2 C-terminal and 2 N-terminal fingers of the overlapping libraries recognize the same sequences. Common fingers recovered in the “overlap positions” of both libraries were used as guides to design 6-fingered proteins. However, 4-fingered proteins recovered to bind the 12-nt target proximal to the cut site can also be used directly. In this way, both 4 and 6-fingered proteins were selected using the counter selection assay detailed above.

Two 4 and two 6-fingered proteins that target the shifted CCR5 sequence were tested for their function as ZFNs in K562 cells. These fingers are related in that the 4 C-terminal fingers of the 6-fingered proteins are identical to one of the 4-fingered variants. Therefore, any differences in activity are due to the addition of two fingers (FIG. 4B, all zinc finger sequences available in the supplementary information). As above, the indel frequency at both the CCR5 and CCR2 loci were measured for each ZFN pair (FIG. 4C). Interestingly, the number of fingers has a large impact on the right target but not the left. Only ZFNs that utilize right monomers with 6 fingers provide high CCR5 indel frequencies, ranging from 19-61%. What's more, for each of these 8 combinations the CCR2 indel frequency ranges from 0.09-0.14%, similar to the frequency found from a background GFP sample. As a result, we are able to leverage differences in the CCR5 and CCR2 sequences, while remaining in close proximity to the desired cut site, by slightly shifting and expanding our target. By doing so we have created ZFNs that offer strong CCR5 activity and little if any activity at CCR2.

B. HBB

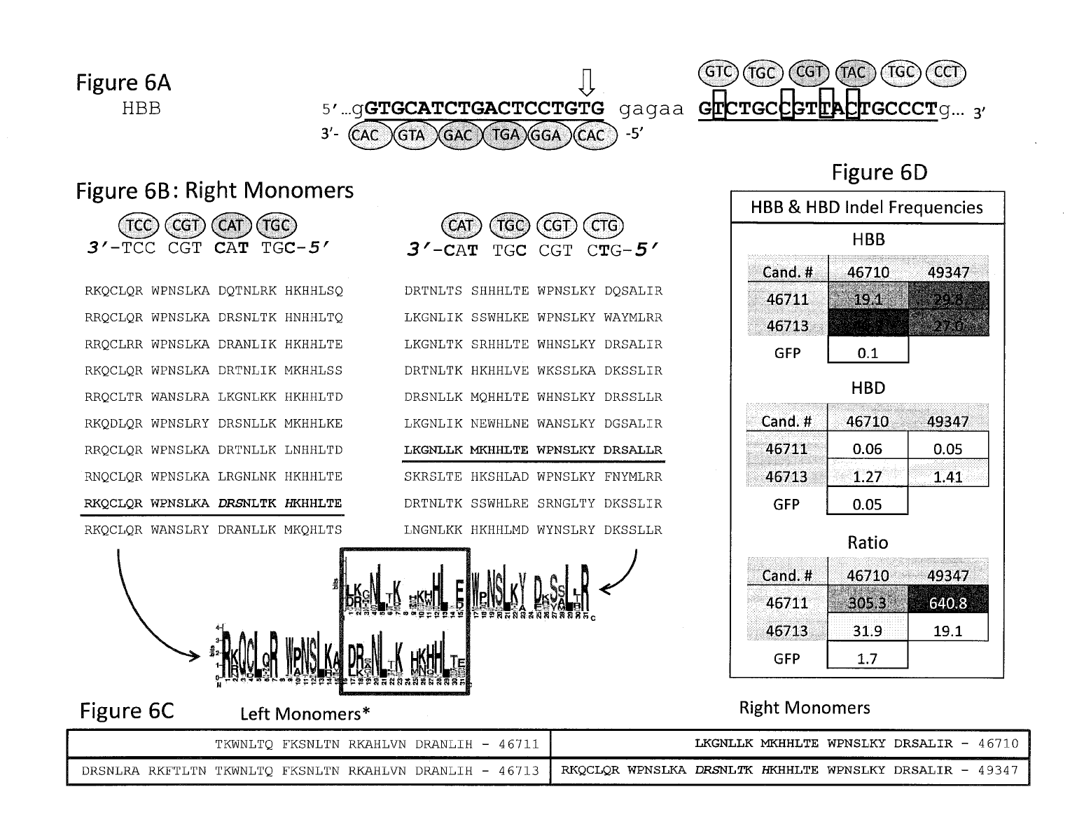

A second disease-related target complicated by the need for discrimination against a highly conserved homolog is Hemoglobin beta (HBB). Mutations in the HBB sequence lead to sickle cell anemia as well as other blood-borne diseases. The coding sequences for HBB and Hemoglobin delta (HBD) are 93% identical in human making it difficult to modify HBB without altering HBD. Using the same approach as outlined above, we focused the selection of zinc fingers to bind directly at a mutant HBB sequence that causes sickle cell anemia (FIG. 6A).

Overlapping 4-finger libraries were created to select 4-finger zinc fingers, and thereby design 6-finger proteins, that can discriminate between HBB and HBD using the same approach detailed above (selected ZFPs shown in FIG. 6B). From these results, a 4 and 6 finger protein that bind the right and left targets were paired (see FIG. 6) and tested as ZFNs in K562 cells. For each pair, indel frequencies were measured at both the HBB and HBD loci (FIG. 6C).

Interestingly, while these ZFNs produce high HBB indel frequencies regardless of finger number, the HBD indel frequency is largely dependent on the number of fingers in the left monomer. In both cases, the 6-finger left monomers increase HBD indel frequencies from background levels to low percentages. As the extension of the left monomer does not pick up additional differences between the HBB and HBD sequences, it is possible the extended recognition increases the affinity at the HBD target while lessoning the consequence of the single mismatch present in the left monomer targets.

These results show that the extension of a nuclease target that does not increase the number of mismatches relative to a similar sequence in the genome may lead to an increase in off-target activity. Regardless, we have produced ZFNs that target the sickle cell mutation in HBB with high activity.

C. Zinc Finger Proteins

Table A shows a number of zinc finger binding domains as described herein. Each row describes a separate zinc finger DNA-binding domain. The DNA target sequence for each domain is shown in the first column (DNA target sites indicated in uppercase letters; non-contacted nucleotides indicated in lowercase), and the remaining columns show the amino acid sequence of the recognition region (amino acids −1 through +6, with respect to the start of the helix) of each of the zinc fingers (F1 through F4 or F1 for four-finger proteins to F6 for six-finger proteins) in the protein. Also provided in the first column is an identification number for each protein.

TABLE A
Zinc finger nuclease designs

| | Design | | | | | |
|---|---|---|---|---|---|---|
| SBS #, Target | F1 | F2 | F3 | F4 | F5 | F6 |

CCR5-specific designs

| | | | | | | |
|---|---|---|---|---|---|---|
| SBS#8266 | DRSNLSR | ISSNLNS | RSDNLAR | TSGNLTR | N/A | N/A |
| GATGAGGATGAC | (SEQ ID | (SEQ ID | (SEQ ID | (SEQ ID | | |
| (SEQ ID | NO: 34) | NO: 35) | NO: 36) | NO: 37) | | |
| NO: 28) | | | | | | |
| SBS#20505 | RSDNLSV | QKINLQV | RSDVLSE | QRNHRTT | N/A | N/A |
| AAACTGCAAAAG | (SEQ ID | (SEQ ID | (SEQ ID | (SEQ ID | | |
| (SEQ ID | NO: 38) | NO: 39) | NO: 40) | NO: 41) | | |
| NO: 29) | | | | | | |
| SBS#46693 | DQSNLTR | APSNLWR | TLYNLTR | FLGNLTR | N/A | N/A |
| agGATGAGGATGA | (SEQ ID | (SEQ ID | (SEQ ID | (SEQ ID | | |
| Ccagcatgttgtt | NO: 42) | NO: 43) | NO: 44) | NO: 45) | | |
| gc (SEQ ID | | | | | | |
| NO: 30) | | | | | | |
| SBS#46696 | TKWNLTT | QKINLTA | RKWVLDC | NPGSLHN | N/A | N/A |
| atAAACTGCAAAA | (SEQ ID | (SEQ ID | (SEQ ID | (SEQ ID | | |
| Ggctgaagagcat | NO: 46) | NO: 47) | NO: 48) | NO: 49) | | |
| ga (SEQ ID | | | | | | |
| NO: 31) | | | | | | |
| SBS#46697 | TKWNLTT | QKINLTA | RKSTLND | QKGNLNQ | N/A | N/A |
| atAACTGCAAAA | (SEQ ID | (SEQ ID | (SEQ ID | (SEQ ID | | |
| Ggctgaagagcat | NO: 46) | NO: 47) | NO: 50) | NO: 51) | | |
| ga (SEQ ID | | | | | | |
| NO: 31) | | | | | | |
| SBS#46698 | RKAHLVN | WQSGLCN | RKSHLVD | SASGLCH | N/A | N/A |
| ttATCAGGATGAG | (SEQ ID | (SEQ ID | (SEQ ID | (SEQ ID | | |
| Gatgaccagcatg | NO: 52) | NO: 53) | NO: 54) | NO: 55) | | |
| tt (SEQ ID | | | | | | |
| NO: 32) | | | | | | |
| SBS#46705 | TKQNLTH | SLFNLKR | QLCNLIR | LKSTLVN | RKDNLKS | QKINLVN |
| tgCAAAAGGCTGA | (SEQ ID | (SEQ ID | (SEQ ID | (SEQ ID | (SEQ ID | (SEQ ID |
| AGAGCATgactga | NO: 56) | NO: 57) | NO: 58) | NO: 59) | NO: 60) | NO: 61) |
| ca(SEQ ID | | | | | | |
| NO: 33) | | | | | | |
| SBS#46700 | DPRSLVN | ARNGLWQ | RKAHLVN | WQSGLCN | RKSHLVD | SASGLCH |
| ttATCAGGATGAG | (SEQ ID | (SEQ ID | (SEQ ID | (SEQ ID | (SEQ ID | (SEQ ID |
| GATGACCAgcatg | NO: 62) | NO: 63) | NO: 52) | NO: 53) | NO: 54) | NO: 55) |
| tt (SEQ ID | | | | | | |
| NO: 32) | | | | | | |
| SBS#46703 | QLCNLIR | LKSTLVN | RKDNLKS | QKINLVN | N/A | N/A |
| tgCAAAAGGCTGA | (SEQ ID | (SEQ ID | (SEQ ID | (SEQ ID | | |
| Agagcatgactga | NO: 58) | NO: 59) | NO: 60) | NO: 61) | | |
| ca (SEQ ID | | | | | | |
| NO: 33) | | | | | | |
| SBS#44671 | TKWNLDT | RASTLWH | RKSTLVE | QRGNLKT | N/A | N/A |
| atAAACTGCAAAA | (SEQ ID | (SEQ ID | (SEQ ID | (SEQ ID | | |
| Ggctgaagag | NO: 137) | NO: 138) | NO: 139) | NO: 140) | | |
| (SEQ ID | | | | | | |
| NO: 141) | | | | | | |
| SBS#44672 | DRSNLLR | FLGNLRR | TQFNLER | MRANLRR | N/A | N/A |
| agGATGAGGATGA | (SEQ ID | (SEQ ID | (SEQ ID | (SEQ ID | | |
| Ccagcatgtt | NO: 143) | NO: 144) | NO: 145) | NO: 146) | | |
| (SEQ ID | | | | | | |
| NO: 142) | | | | | | |
| SBS#46694 | WQANLLR | FASNLIR | TLWSLTR | TKQNLQR | N/A | N/A |
| agGATGAGGATGA | (SEQ ID | (SEQ ID | (SEQ ID | (SEQ ID | | |
| Ccagcatgttgtt | NO: 147) | NO: 148) | NO: 149) | NO: 150) | | |
| gc (SEQ ID | | | | | | |
| NO: 30) | | | | | | |
| SBS#46695 | RKDNLTQ | RASTLWH | RKSTLND | QKGNLNQ | N/A | N/A |
| atAAACTGCAAAA | (SEQ ID | (SEQ ID | (SEQ ID | (SEQ ID | | |
| Ggctgaagagcat | NO: 151) | NO: 138) | NO: 50) | NO: 51) | | |
| ga (SEQ ID | | | | | | |
| NO: 31) | | | | | | |
| SBS#46699 | RKAHLVD | ARAGLWQ | RKANLYN | SQSGLCH | N/A | N/A |
| ttATCAGGATGAG | (SEQ ID | (SEQ ID | (SEQ ID | (SEQ ID | | |
| Gatgaccagcatg | NO: 152) | NO: 153) | NO: 154) | NO: 155) | | |
| tt (SEQ ID | | | | | | |
| NO: 32) | | | | | | |
| SBS#46701 | EKRGLLN | SGAGLWQ | RKAHLVD | ARAGLWQ | RKANLYN | SQSGLCH |
| ttATCAGGATGAG | (SEQ ID | (SEQ ID | (SEQ ID | (SEQ ID | (SEQ ID | (SEQ ID |
| GATGACCAgcatg | NO: 156) | NO: 157) | NO: 152) | NO: 153) | NO: 154) | NO: 155) |
| tt (SEQ ID | | | | | | |
| NO: 32) | | | | | | |
| SBS#46702 | AQSNLLR | RKPDLVR | RKDNLRD | QKINLNQ | N/A | N/A |
| tgCAAAAGGCTGA | (SEQ ID | (SEQ ID | (SEQ ID | (SEQ ID | | |
| Agagcatgactga | NO: 158) | NO: 159) | NO: 160) | NO: 161) | | |
| ca (SEQ ID | | | | | | |
| NO: 33) | | | | | | |
| SBS#46703 | QLCNLIR | LKSTLVN | RKDNLKS | QKINLVN | N/A | N/A |
| tgCAAAAGGCTGA | (SEQ ID | (SEQ ID | (SEQ ID | (SEQ ID | | |
| Agagcatgactga | NO: 58) | NO: 59) | NO: 60) | NO: 61) | | |
| ca (SEQ ID | | | | | | |
| NO: 33) | | | | | | |
| SBS#46704 | TKQNLQT | TLFNLTR | AQSNLLR | RKPDLVR | RKDNLRD | QKINLNQ |
| tgCAAAAGGCTGA | (SEQ ID | (SEQ ID | (SEQ ID | (SEQ ID | (SEQ ID | (SEQ ID |
| AGAGCATgactga | NO: 162) | NO: 163) | NO: 158) | NO: 159) | NO: 160) | NO: 161) |
| ca (SEQ ID | | | | | | |
| NO: 33) | | | | | | |
| ZFN NS | RKDNLRT | QKGNLNS | RKSTLND | QKGNLNQ | N/A | N/A |
| atAAACTGCAAAA | (SEQ ID | (SEQ ID | (SEQ ID | (SEQ ID | | |
| Ggctgaagagcat | NO: 64) | NO: 65) | NO: 50) | NO: 51) | | |
| ga (SEQ ID | | | | | | |
| NO: 31) | | | | | | |

Hbb Designs:

| | | | | | | |
|---|---|---|---|---|---|---|
| ZFN A | RKQCLQR | WPNSLKA | DQTNLRK | HKHHLSQ | N/A | N/A |
| TCCCGTCATTGC | (SEQ ID | (SEQ ID | (SEQ ID | (SEQ ID | | |
| (SEQ ID | NO: 67) | NO: 68) | NO: 69) | NO: 70) | | |
| NO: 66) | | | | | | |
| ZFN B | RRQCLQR | WPNSLKA | DRSNLTK | HNHHLTQ | N/A | N/A |
| TCCCGTCATTGC | (SEQ ID | (SEQ ID | (SEQ ID | (SEQ ID | | |
| (SEQ ID | NO: 71) | NO: 68) | NO: 72 | NO: 73) | | |
| NO: 66) | | | | | | |
| ZFN C | RRQCLRR | WPNSLKA | DRANLIK | HKHHLTE | N/A | N/A |
| TCCCGTCATTGC | (SEQ ID | (SEQ ID | (SEQ ID | (SEQ ID | | |
| (SEQ ID | NO: 74) | NO: 68) | NO: 75) | NO: 76) | | |
| NO: 66) | | | | | | |
| ZFN D | RKQCLQR | WPNSLKA | DRTNLIK | MKHHLSS | N/A | N/A |
| TCCCGTCATTGC | (SEQ ID | (SEQ ID | (SEQ ID | (SEQ ID | | |
| (SEQ ID | NO: 67) | NO: 68) | NO: 77) | NO: 78) | | |
| NO: 66) | | | | | | |
| ZFN E | RRQCLTR | WANSLRA | LKGNLKK | HKHHLTD | N/A | N/A |
| TCCCGTCATTGC | (SEQ ID | (SEQ ID | (SEQ ID | (SEQ ID | | |
| (SEQ ID | NO: 79) | NO: 80) | NO: 81) | NO: 82) | | |
| NO: 66) | | | | | | |
| ZFN F | RKQDLQR | WPNSLRY | DRSNLLK | MKHHLKE | N/A | N/A |
| TCCCGTCATTGC | (SEQ ID | (SEQ ID | (SEQ ID | (SEQ ID | | |
| (SEQ ID | NO: 83) | NO: 84) | NO: 85) | NO: 86) | | |
| NO: 66) | | | | | | |
| ZFN G | RRQCLQR | WPNSLKA | DRTNLLK | LNHHLTD | N/A | N/A |
| TCCCGTCATTGC | (SEQ ID | (SEQ ID | (SEQ ID | (SEQ ID | | |
| (SEQ ID | NO: 71) | NO: 68) | NO: 87) | NO: 88) | | |
| NO: 66) | | | | | | |
| ZFN H | RNQCLQR | WPNSLKA | LRGNLNK | HKHHLTE | N/A | N/A |
| TCCCGTCATTGC | (SEQ ID | (SEQ ID | (SEQ ID | (SEQ ID | | |
| (SEQ ID | NO: 89) | NO: 68) | NO: 90) | NO: 76) | | |
| NO: 66) | | | | | | |
| ZFN I | RKQCLQR | WPNSLKA | DRSNLTK | HKHHLTE | N/A | N/A |
| TCCCGTCATTGC | (SEQ ID | (SEQ ID | (SEQ ID | (SEQ ID | | |
| (SEQ ID | NO: 67) | NO: 68) | NO: 72) | NO: 76) | | |
| NO: 66) | | | | | | |
| ZFN J | RKQCLQR | WANSLRY | DRANLLK | MKQHLTS | N/A | N/A |
| TCCCGTCATTGC | (SEQ ID | (SEQ ID | (SEQ ID | (SEQ ID | | |
| (SEQ ID | NO: 67) | NO: 91) | NO: 92) | NO: 93) | | |
| NO: 66) | | | | | | |
| ZFN K | DRTNLTS | SHHHLTE | WPNSLKY | DQSALIR | N/A | N/A |
| CATTGCCGTCTG | (SEQ ID | (SEQ ID | (SEQ ID | (SEQ ID | | |
| (SEQ ID | NO: 95) | NO: 96) | NO: 97) | NO: 98) | | |
| NO: 94) | | | | | | |
| ZFN L | LKGNLIK | SSWHLKE | WPNSLKY | WAYMLRR | N/A | N/A |
| CATTGCCGTCTG | (SEQ ID | (SEQ ID | (SEQ ID | (SEQ ID | | |
| (SEQ ID | NO: 99) | NO: 100 | NO: 97) | NO: 101) | | |
| NO: 94) | | | | | | |
| ZFN M | LKGNLTK | SRHHLTE | WHNSLKY | DRSALIR | N/A | N/A |
| CATTGCCGTCTG | (SEQ ID | (SEQ ID | (SEQ ID | (SEQ ID | | |
| (SEQ ID | NO: 102) | NO: 103) | NO: 104) | NO: 105) | | |
| NO: 94) | | | | | | |
| ZFN N | DRTNLTK | HKHHLVE | WKSSLKA | DKSSLIR | N/A | N/A |
| CATTGCCGTCTG | (SEQ ID | (SEQ ID | (SEQ ID | (SEQ ID | | |
| (SEQ ID | NO: 106) | NO: 107) | NO: 108) | NO: 109) | | |
| NO: 94) | | | | | | |
| ZFN O | DRSNLLK | MQHHLTE | WHNSLKY | DRSSLLR | N/A | N/A |
| CATTGCCGTCTG | (SEQ ID | (SEQ ID | (SEQ ID | (SEQ ID | | |
| (SEQ ID | NO: 85) | NO: 110) | NO: 104) | NO: 111) | | |
| NO: 94) | | | | | | |
| ZFN P | LKGNLIK | NEWHLNE | WANSLKY | DGSALIR | N/A | N/A |
| CATTGCCGTCTG | (SEQ ID | (SEQ ID | (SEQ ID | (SEQ ID | | |
| (SEQ ID | NO: 99) | NO: 112) | NO: 113) | NO: 114) | | |
| NO: 94) | | | | | | |
| ZFN Q | LKGNLLK | MKHHLTE | WPNSLKY | DRSALLR | N/A | N/A |
| CATTGCCGTCTG | (SEQ ID | (SEQ ID | (SEQ ID | (SEQ ID | | |
| (SEQ ID | NO: 115) | NO: 116) | NO: 97) | NO: 117) | | |
| NO: 94) | | | | | | |
| ZFN R | SKRSLTE | HKSHLAD | WPNSLKY | FNYMLRR | N/A | N/A |
| CATTGCCGTCTG | (SEQ ID | (SEQ ID | (SEQ ID | (SEQ ID | | |
| (SEQ ID | NO: 118) | NO: 119) | NO: 97) | NO: 120) | | |
| NO: 94) | | | | | | |
| ZFN S | DRTNLTK | SSWHLRE | SRNGLTY | DKSSLIR | N/A | N/A |
| CATTGCCGTCTG | (SEQ ID | (SEQ ID | (SEQ ID | (SEQ ID | | |
| (SEQ ID | NO: 106 | NO: 121) | NO: 122) | NO: 109) | | |
| NO: 94) | | | | | | |
| ZFN T | LNGNLKK | HKHHLMD | WYNSLRY | DKSSLLR | N/A | N/A |
| CATTGCCGTCTG | (SEQ ID | (SEQ ID | (SEQ ID | (SEQ ID | | |
| (SEQ ID | NO: 123) | NO: 124) | NO: 125) | NO: 126) | | |
| NO: 94) | | | | | | |
| SBS#46711 | TKWNLTQ | FKSNLTN | RKAHLVN | DRANLIH | N/A | N/A |
| tcCACAGGAGTCA | (SEQ ID | (SEQ ID | (SEQ ID | (SEQ ID | | |
| Gatgcaccatggt | NO: 130) | NO: 131) | NO: 132) | NO: 133) | | |
| gt (SEQ ID | | | | | | |
| NO: 127) | | | | | | |
| SBS#46713 | DRSNLRA | RKFTLTN | TKWNLTQ | FKSNLTN | RKAHLVN | DRANLIH |
| tcCACAGGAGTCA | (SEQ ID | (SEQ ID | (SEQ ID | (SEQ ID | (SEQ ID | (SEQ ID |
| GATGCACcatggt | NO: 134) | NO: 135) | NO: 130) | NO: 131) | NO: 132) | NO: 133) |
| gt (SEQ ID | | | | | | |
| NO: 127) | | | | | | |
| SBS#46710 | LKGNLLK | MKHHLTE | WPNSLKY | DRSALIR | N/A | N/A |
| agTCTGCCGTTAC | (SEQ ID | (SEQ ID | (SEQ ID | (SEQ ID | | |
| Tgccctgtggggc | NO: 115) | NO: 116) | NO: 97) | NO: 105) | | |
| aa (SEQ ID | | | | | | |
| NO: 128) | | | | | | |
| SBS#49347 | RKQCLQR | WPNSLKA | DRSNLTK | HKHHLTE | WPNSLKY | DRSALIR |
| aaGTCTGCCGTTA | (SEQ ID | (SEQ ID | (SEQ ID | (SEQ ID | (SEQ ID | (SEQ ID |
| CTGCCCTgtgggg | NO: 67) | NO: 68) | NO: 72) | NO: 76) | NO: 97) | NO: 105) |
| ca | | | | | | |
| (SEQ ID | | | | | | |
| NO: 129) | | | | | | |
| ZFN AA | DRSNLRA | RNSTLIE | RKDNLTQ | FKSNLTS | N/A | N/A |
| AGTCAGATGCAC | (SEQ ID | (SEQ ID | (SEQ ID | (SEQ ID | | |
| (SEQ ID | NO: 166) | NO: 167) | NO: 168) | NO: 169) | | |
| NO: 164) | | | | | | |
| ZFN BB | DQSNLTR | RKFTLTN | RKGNLKE | FHSNLIA | N/A | N/A |
| AGTCAGATGCAC | (SEQ ID | (SEQ ID | (SEQ ID | (SEQ ID | | |
| (SEQ ID | NO: 42) | NO: 170) | NO: 171) | NO: 172) | | |
| NO: 164) | | | | | | |
| ZFN CC | DRSNLRA | RKSTLRL | RKGNLNS | WKSNLTS | N/A | N/A |
| AGTCAGATGCAC | (SEQ ID | (SEQ ID | (SEQ ID | (SEQ ID | | |
| (SEQ ID | NO: 166) | NO: 173) | NO: 174) | NO: 175) | | |
| NO: 164) | | | | | | |
| ZFN DD | LKGNLRQ | RKSTLRL | RKANLRD | FHSNLIA | N/A | N/A |
| AGTCAGATGCAC | (SEQ ID | (SEQ ID | (SEQ ID | (SEQ ID | | |
| (SEQ ID | NO: 176) | NO: 173) | NO: 177) | NO: 172) | | |
| NO: 164) | | | | | | |
| ZFN EE | DRSNLRH | RKFTLTN | RKGNLKD | MKHHLTD | N/A | N/A |
| AGTCAGATGCAC | (SEQ ID | (SEQ ID | (SEQ ID | (SEQ ID | | |
| (SEQ ID | NO: 178) | NO: 170) | NO: 179) | NO: 180) | | |
| NO: 164) | | | | | | |
| ZFN FF | DQSNLTR | RKFVLTN | TKWNLTQ | WKSNLTN | N/A | N/A |
| AGTCAGATGCAC | (SEQ ID | (SEQ ID | (SEQ ID | (SEQ ID | | |
| (SEQ ID | NO: 42) | NO: 181) | NO: 182) | NO: 183) | | |
| NO: 164) | | | | | | |
| ZFN GG | DRSNLRA | RNSTLIE | RKGNLKD | FKSNLTN | N/A | N/A |
| AGTCAGATGCAC | (SEQ ID | (SEQ ID | (SEQ ID | (SEQ ID | | |
| (SEQ ID | NO: 166) | NO: 167) | NO: 179) | NO: 131) | | |
| NO: 164) | | | | | | |
| ZFN HH | DRSNLRA | RKFTLTN | TKWNLTQ | FKSNLTN | N/A | N/A |
| AGTCAGATGCAC | (SEQ ID | (SEQ ID | (SEQ ID | (SEQ ID | | |
| (SEQ ID | NO: 166) | NO: 170) | NO: 182) | NO: 131) | | |
| NO: 164) | | | | | | |
| ZFN II | DRSNLRH | RKSTLRL | TKWNLTQ | FQSNLTN | N/A | N/A |
| AGTCAGATGCAC | (SEQ ID | (SEQ ID | (SEQ ID | (SEQ ID | | |
| (SEQ ID | NO: 178) | NO: 173) | NO: 182) | NO: 184) | | |
| NO: 164) | | | | | | |
| ZFN JJ | YKRSLVD | RKSTLIE | RKGNLKD | MKHHLTS | N/A | N/A |
| AGTCAGATGCAC | (SEQ ID | (SEQ ID | (SEQ ID | (SEQ ID | | |
| (SEQ ID | NO: 185) | NO: 186) | NO: 179) | NO: 187) | | |
| NO: 164) | | | | | | |
| ZFN KK | TKWNLTQ | FHSNLIA | RKDTLVT | DRSNLIA | N/A | N/A |
| CACAGGAGTCAG | (SEQ ID | (SEQ ID | (SEQ ID | (SEQ ID | | |
| (SEQ ID | NO: 182) | NO: 172) | NO: 188) | NO: 189) | | |
| NO: 165) | | | | | | |
| ZFN LL | RKANLND | WKHVLTN | RKDHLVD | HRSNLIH | N/A | N/A |
| CACAGGAGTCAG | (SEQ ID | (SEQ ID | (SEQ ID | (SEQ ID | | |
| (SEQ ID | NO: 190) | NO: 191) | NO: 192) | NO: 193) | | |
| NO: 165) | | | | | | |
| ZFN MM | TKWNLTQ | LNQHLTN | RKDHLVN | HQSNLIH | N/A | N/A |
| CACAGGAGTCAG | (SEQ ID | (SEQ ID | (SEQ ID | (SEQ ID | | |
| (SEQ ID | NO: 182) | NO: 194) | NO: 195) | NO: 196) | | |
| NO: 165) | | | | | | |
| ZFN NN | TKWNLTQ | WQSNLTE | RKAHLYN | DQANLRH | N/A | N/A |
| CACAGGAGTCAG | (SEQ ID | (SEQ ID | (SEQ ID | (SEQ ID | | |
| (SEQ ID | NO: 182) | NO: 197) | NO: 198) | NO: 199) | | |
| NO: 165) | | | | | | |
| ZFN OO | TKWNLTT | FKSNLTS | RKSHLVD | CKPNLLS | N/A | N/A |
| CACAGGAGTCAG | (SEQ ID | (SEQ ID | (SEQ ID | (SEQ ID | | |
| (SEQ ID | NO: 200) | NO: 169) | NO: 201) | NO: 202) | | |
| NO: 165) | | | | | | |
| ZFN PP | TKWNLTT | LNHHLTQ | RKSHLVD | LKGNLRQ | N/A | N/A |
| CACAGGAGTCAG | (SEQ ID | (SEQ ID | (SEQ ID | (SEQ ID | | |
| (SEQ ID | NO: 200) | NO: 203) | NO: 201) | NO: 176) | | |
| NO: 165) | | | | | | |
| ZFN QQ | TKWNLTQ | LKHHLTN | RKDHLVN | HRSNLIH | N/A | N/A |
| CACAGGAGTCAG | (SEQ ID | (SEQ ID | (SEQ ID | (SEQ ID | | |
| (SEQ ID | NO: 182) | NO: 204) | NO: 195) | NO: 193) | | |
| NO: 165) | | | | | | |
| ZFN RR | RKANLND | FQSNLTN | RKDHLVN | HQANLIH | N/A | N/A |
| CACAGGAGTCAG | (SEQ ID | (SEQ ID | (SEQ ID | (SEQ ID | | |
| (SEQ ID | NO: 190) | NO: 184) | NO: 195) | NO: 205) | | |
| NO: 165) | | | | | | |
| ZFN SS | TKWNLTQ | FHSNLIA | RKDTLVT | DRSNLIA | N/A | N/A |
| CACAGGAGTCAG | (SEQ ID | (SEQ ID | (SEQ ID | (SEQ ID | | |
| (SEQ ID | NO: 182) | NO: 172) | NO: 188) | NO: 189) | | |
| NO: 165) | | | | | | |

### Example 6: An Unbiased Genomic Screen Reveals Negligible Off-Target Activity

The ability of a ZFP to discriminate between closely related sequences is a powerful indicator of in vivo specificity but does not eliminate the possibility that these fine-tuned nucleases bind and modify other, less predictable sequences. In fact, an unbiased screen of genome-wide cutting has shown that substantial indel frequencies can be found at sequences with as little as 67% homology with the desired target, while other highly homologous targets remain untouched, demonstrating that genome-wide fidelity can be more complicated than sequence similarity alone.

Therefore, in order to map off-target loci for our different CCR5 ZFN pairs, we used a modified version of this integrase-defective lentiviral vector (IDLV) capture and mapping protocol from Gabriel et al. (2011) Nat Biotech 29:816-823. To increase the sensitivity of the assay, we used the higher sequencing depth provided by Illumina sequencing and performed the assay in biologic triplicate to minimize apparent clusters of IDLV integrations caused simply by obtaining more sequence reads. We ranked clusters of IDLV integrations (IDLV integrations within 1,000 base pairs of other IDLV integrations) based on the total number of integrations, the number of replicates containing those integrations, and the ratio of integrations in the ZFN treated samples to the control samples (see Example 1).

We then designed PCR primers to amplify the top 20 ranked clusters for each ZFN pair and characterized the indel frequency at each of these 20 potential off-target site per ZFN pair; to provide additional information we also characterized indel frequencies at loci corresponding to top 20 ranked clusters for other CCR5 ZFNs that target similar sequences (i.e. we tested the activity of 46698:46705 at clusters obtained with either 46698:46705 or 46700:46705). Results are shown in Tables 1A and 1B below.

TABLE 1A
6 Finger ZFN pairs: Off-target activity

| | Genome | | 46698_46705 | | | | 46700_46705 | | | | GFP | | |
|---|---|---|---|---|---|---|---|---|---|---|---|---|---|
| Gene | coordinates | | Total | Indel | % Indel | pval | Total | Indel | % Indel | pval | Total | Indel | % Indel |
| CCR5 | chr3 | 46414562 | 11739 | 2788 | 23.75 | 0 | 14773 | 4040 | 27.35 | 0 | 14738 | 4 | 0.03 |
| | chr4 | 55104705 | 17890 | 160 | 0.89 | 0 | 22680 | 85 | 0.37 | 0 | 18720 | 5 | 0.03 |
| | chr6 | 50841937 | 37492 | 127 | 0.34 | 0 | 20508 | 12 | 0.06 | 1 | 36292 | 15 | 0.04 |
| | chr3 | 45874336 | 38425 | 79 | 0.21 | 0 | 33033 | 33 | 0.1 | 0.0001 | 49044 | 9 | 0.02 |
| | chr22 | 16872698 | 3116 | 2 | 0.06 | 1 | 41689 | 2 | 0 | 1 | 46303 | 12 | 0.03 |
| | chr16 | 3961383 | 25459 | 15 | 0.06 | 1 | 22409 | 12 | 0.05 | 1 | 29704 | 13 | 0.04 |
| | chr13 | 106109633 | 16487 | 9 | 0.05 | 1 | 22460 | 16 | 0.07 | 1 | 13657 | 6 | 0.04 |
| | chr2 | 37418423 | 11282 | 6 | 0.05 | 1 | 7152 | 2 | 0.03 | 1 | 12997 | 3 | 0.02 |
| | chr1 | 1247311 | 14870 | 6 | 0.04 | 1 | 14454 | 6 | 0.04 | 1 | 20175 | 6 | 0.03 |
| | chr17 | 61360858 | 21733 | 8 | 0.04 | 1 | 23163 | 13 | 0.06 | 1 | 6371 | 2 | 0.03 |
| | chr11 | 66963797 | 19628 | 7 | 0.04 | 1 | 19605 | 6 | 0.03 | 1 | 21233 | 5 | 0.02 |
| | chr1 | 164282131 | 27562 | 9 | 0.03 | 1 | 18325 | 11 | 0.06 | 1 | 21678 | 13 | 0.06 |
| | chr1 | 156083765 | 19155 | 6 | 0.03 | 1 | 18227 | 9 | 0.05 | 1 | 20363 | 4 | 0.02 |
| | chr1 | 117547662 | 23166 | 7 | 0.03 | 1 | ND | ND | ND | ND | 22711 | 14 | 0.06 |
| | chr1 | 24397474 | 11466 | 3 | 0.03 | 1 | 3326 | 1 | 0.03 | 1 | 6048 | 0 | 0 |
| | chr4 | 128156339 | 41559 | 9 | 0.02 | 0.038 | 37972 | 11 | 0.03 | 0.0123 | 13242 | 0 | 0 |
| | chr19 | 55627065 | 13859 | 3 | 0.02 | 1 | 16989 | 3 | 0.02 | 1 | 26312 | 10 | 0.04 |
| | chr18 | 24550225 | 26324 | 5 | 0.02 | 1 | 19370 | 6 | 0.03 | 1 | 19576 | 6 | 0.03 |
| | chr14 | 73024112 | 18879 | 3 | 0.02 | 1 | 19064 | 8 | 0.04 | 0.9396 | 10254 | 1 | 0.01 |
| | chr2 | 235655882 | 33073 | 5 | 0.02 | 1 | 34356 | 5 | 0.01 | 1 | 41023 | 7 | 0.02 |
| | chr18 | 37289114 | 18510 | 2 | 0.01 | 1 | 21095 | 7 | 0.03 | 1 | 21657 | 6 | 0.03 |
| | chr11 | 46466237 | 24422 | 2 | 0.01 | 1 | 31927 | 2 | 0.01 | 1 | 19163 | 2 | 0.01 |
| | chr3 | 166220797 | 45346 | 2 | 0 | 1 | 41793 | 2 | 0 | 1 | 51370 | 10 | 0.02 |
| | chr10 | 125317862 | 17850 | 0 | 0 | 1 | 26715 | 4 | 0.01 | 1 | 31672 | 20 | 0.06 |
| | chr5 | 31657118 | 27358 | 0 | 0 | NA | 33571 | 0 | 0 | NA | 47286 | 0 | 0 |
| | chr8 | 12643910 | 9980 | 0 | 0 | 1 | 12894 | 5 | 0.04 | 1 | 10047 | 1 | 0.01 |
| | chr21 | 17633056 | 3835 | 0 | 0 | NA | 4258 | 0 | 0 | NA | 5596 | 0 | 0 |
| | chr13 | 19198322 | 50630 | 0 | 0 | NA | 49837 | 0 | 0 | NA | 65920 | 0 | 0 |
| CCR2 | chr3 | 46399221 | 1396 | 0 | 0 | 1 | 1316 | 1 | 0.08 | 1 | 1878 | 1 | 0.05 |
| | chr1 | 181394105 | ND | ND | ND | ND | ND | ND | ND | ND | ND | ND | ND |
| | chr2 | 231317526 | ND | ND | ND | ND | ND | ND | ND | ND | ND | ND | ND |
| | chrX | 53798450 | ND | ND | ND | ND | ND | ND | ND | ND | ND | ND | ND |

| | | |
|---|---|---|
| Aggregate off- | 1.46 | 0.5 |
| target | | |

TABLE 1B
4 Fingered ZFN: Off target activity

| | Genome | | 8266_20505 | | | | 46693_46696 | | | |
|---|---|---|---|---|---|---|---|---|---|---|
| Gene | coordinates | | Total | Indel | % Indel | pval | Total | Indel | % Indel | pval |
| CCR5 | chr3 | 46414562 | 15368 | 4599 | 29.93 | 0 | 15162 | 632 | 417 | 0 |
| KAR1 | chr12 | 75963464 | 33425 | 4129 | 12.35 | 0 | 51126 | 35 | 0.07 | 0 |
| FBLX11 | chr11 | 66963797 | 38846 | 2175 | 5.6 | 0 | 25303 | 18 | 0.07 | 1 |
| CCR2 | chr3 | 46399221 | 29494 | 1542 | 5.23 | 0 | 71349 | 9 | 0.01 | 1 |
| ZCCH14C | chr16 | 87499226 | 36262 | 1105 | 3.05 | 0 | 18596 | 1 | 0.01 | 1 |
| | chr12 | 22784040 | 65082 | 1663 | 2.56 | 0 | 36444 | 27 | 0.07 | 0.038 |
| | chr21 | 46444698 | 70386 | 1750 | 2.49 | 0 | 61505 | 4 | 0.01 | 1 |
| | chr5 | 141607241 | 37758 | 576 | 1.53 | 0 | 54726 | 30 | 0.05 | 0.0154 |
| | chr22 | 29552889 | 54395 | 213 | 0.39 | 0 | 11028 | 2 | 0.02 | 1 |
| | chr3 | 3129932 | 27052 | 31 | 0.11 | 8E−04 | 27143 | 184 | 0.68 | 0 |
| | chr2 | 7792528 | 35296 | 22 | 0.06 | 0.11 | 32931 | 39 | 0.12 | 0 |
| | chr8 | 125906764 | 30117 | 14 | 0.05 | 1 | 24713 | 8 | 0.03 | 1 |
| | chr19 | 55627065 | 33196 | 15 | 0.05 | 1 | 19219 | 6 | 0.03 | 1 |
| | chr17 | 2222575 | 38528 | 17 | 0.04 | 1 | 16864 | 16 | 0.09 | 0.2835 |
| | chr1 | 24397474 | 11393 | 5 | 0.04 | 1 | 26416 | 15 | 0.06 | 0.9893 |
| | chr9 | 139310747 | 32288 | 14 | 0.04 | 1 | 36196 | 9 | 0.02 | 1 |
| | chr2 | 108698300 | 37696 | 16 | 0.04 | 1 | 21707 | 15 | 0.07 | 1 |
| | chr11 | 46466237 | 21251 | 9 | 0.04 | 1 | 29923 | 1636 | 5.47 | 0 |
| | chr1 | 33031423 | 33874 | 14 | 0.04 | 1 | 21545 | 15 | 0.07 | 0.4201 |
| | chr11 | 33210103 | 45602 | 18 | 0.04 | 1 | 33069 | 9 | 0.03 | 1 |
| | chr2 | 11466657 | 34495 | 13 | 0.04 | 1 | 47272 | 733 | 1.55 | 0 |
| | chr6 | 62730342 | 44876 | 16 | 0.04 | 1 | 1382 | 2 | 0.14 | 1 |
| | chr17 | 61360858 | 40697 | 14 | 0.03 | 1 | 28602 | 10 | 0.03 | 1 |
| | chr18 | 39489523 | 15412 | 5 | 0.03 | 1 | 66905 | 77 | 0.12 | 0.0001 |
| | chr3 | 49159926 | 34172 | 11 | 0.03 | 1 | 37954 | 11 | 0.03 | 1 |
| | chr4 | 19016245 | 40975 | 13 | 0.03 | 1 | 43031 | 12 | 0.03 | 1 |
| | chr4 | 70465910 | 42283 | 13 | 0.03 | 1 | 2666 | 0 | 0 | 1 |
| | chr5 | 104163083 | 48271 | 14 | 0.03 | 1 | 24838 | 13 | 0.05 | 1 |
| | chr14 | 107044711 | 47321 | 12 | 0.03 | 1 | 24040 | 2144 | 8.92 | 0 |
| | chr19 | 14634430 | 4017 | 1 | 0.02 | 1 | 3844 | 1 | 0.03 | 1 |
| | chr14 | 73024112 | 20814 | 5 | 0.02 | 1 | 35314 | 842 | 2.38 | 0 |
| | chr2 | 37418423 | 4382 | 1 | 0.02 | 1 | 4606 | 5 | 0.11 | 1 |
| | chr1 | 1247311 | 14150 | 3 | 0.02 | 1 | 33664 | 15 | 0.04 | 1 |
| | chr2 | 106258357 | 26544 | 5 | 0.02 | 1 | 25495 | 440 | 1.73 | 0 |
| | chr11 | 5248224 | 24662 | 3 | 0.01 | 1 | 43067 | 5 | 0.01 | 1 |
| | chr4 | 140537415 | 48768 | 5 | 0.01 | 1 | 31581 | 3 | 0.01 | 1 |
| | chr1 | 14704357 | 2963 | 0 | 0 | 1 | 19739 | 0 | 0 | 1 |
| | chr12 | 33171137 | 5695 | 0 | 0 | 1 | 5473 | 0 | 0 | 1 |
| | chr10 | 42534687 | 33937 | 0 | 0 | 0 | NA | 40362 | 0 | NA |
| | chr11 | 24428875 | 1988 | 0 | 0 | 1 | ND | ND | ND | ND |
| | chr3 | 23606938 | ND | ND | ND | ND | ND | ND | ND | ND |
| | chrX | 53798450 | ND | ND | ND | ND | ND | ND | ND | ND |
| | chr5 | 145526114 | ND | ND | ND | ND | ND | ND | ND | ND |
| | chr7 | 80399197 | ND | ND | ND | ND | ND | ND | ND | ND |
| | chr1 | 199403463 | ND | ND | ND | ND | ND | ND | ND | ND |

| | | |
|---|---|---|
| Aggregate off-target | 33.3 | 21.2 |

| | Genome | | 46693_46697 | | | | GFP | | |
|---|---|---|---|---|---|---|---|---|---|
| Gene | coordinates | | Total | Indel | % Indel | pval | Total | Indel | % Indel |
| CCR5 | chr3 | 46414562 | 16699 | 1672 | 10.01 | 0 | 14738 | 1 | 0.01 |
| KAR1 | chr12 | 75963464 | 40581 | 37 | 0.09 | 0 | 2411 | 0 | 0 |
| FBLX11 | chr11 | 66963797 | 22036 | 7 | 0.03 | 1 | 25890 | 11 | 0.04 |
| CCR2 | chr3 | 46399221 | 43318 | 29 | 0.07 | 0.0013 | 45993 | 6 | 0.01 |
| ZCCH14C | chr16 | 87499226 | 21243 | 7 | 0.03 | 1 | 18913 | 7 | 0.04 |
| | chr12 | 22784040 | 37908 | 12 | 0.03 | 1 | 30854 | 7 | 0.02 |
| | chr21 | 46444698 | 58028 | 6 | 0.01 | 1 | 44081 | 5 | 0.01 |
| | chr5 | 141607241 | 42465 | 17 | 0.04 | 0.5552 | 28650 | 4 | 0.01 |
| | chr22 | 29552889 | 41627 | 12 | 0.03 | 1 | 31633 | 4 | 0.01 |
| | chr3 | 3129932 | 26157 | 38 | 0.15 | 0 | 27119 | 6 | 0.02 |
| | chr2 | 7792528 | 17003 | 8 | 0.05 | 1 | 13224 | 2 | 0.02 |
| | chr8 | 125906764 | 20144 | 33 | 0.16 | 0.0187 | 10621 | 5 | 0.05 |
| | chr19 | 55627065 | 17716 | 3 | 0.02 | 1 | 19813 | 5 | 0.03 |
| | chr17 | 2222575 | 22005 | 23 | 0.1 | 0.0522 | 22842 | 7 | 0.03 |
| | chr1 | 24397474 | 13458 | 108 | 0.8 | 0 | 18619 | 4 | 0.02 |
| | chr9 | 139310747 | 22417 | 8 | 0.04 | 1 | 21127 | 10 | 0.05 |
| | chr2 | 108698300 | 22378 | 38 | 0.17 | 0.0005 | 30011 | 13 | 0.04 |
| | chr11 | 46466237 | 23051 | 307 | 1.33 | 0 | 24591 | 5 | 0.02 |
| | chr1 | 33031423 | 27118 | 11 | 0.04 | 1 | 22229 | 5 | 0.02 |
| | chr11 | 33210103 | 22676 | 8 | 0.04 | 1 | 23677 | 4 | 0.02 |
| | chr2 | 11466657 | 25773 | 19 | 0.07 | 1 | 21805 | 9 | 0.04 |
| | chr6 | 62730342 | 30195 | 5 | 0.02 | 1 | 27394 | 12 | 0.04 |
| | chr17 | 61360858 | 24156 | 7 | 0.03 | 1 | 29142 | 4 | 0.01 |
| | chr18 | 39489523 | 39133 | 9 | 0.02 | 1 | 40787 | 16 | 0.04 |
| | chr3 | 49159926 | 24153 | 8 | 0.03 | 1 | 16248 | 2 | 0.01 |
| | chr4 | 19016245 | 27456 | 19 | 0.07 | 1 | 21825 | 9 | 0.04 |
| | chr4 | 70465910 | 22360 | 11 | 0.05 | 1 | 6388 | 2 | 0.03 |
| | chr5 | 104163083 | 23482 | 8 | 0.03 | 1 | 16770 | 6 | 0.04 |
| | chr14 | 107044711 | 12877 | 6 | 0.05 | 1 | 32827 | 7 | 0.02 |
| | chr19 | 14634430 | 4590 | 2 | 0.04 | 1 | 2984 | 2 | 0.07 |
| | chr14 | 73024112 | 22175 | 3 | 0.01 | 1 | 22978 | 3 | 0.01 |
| | chr2 | 37418423 | 10898 | 3 | 0.03 | 1 | 9238 | 3 | 0.03 |
| | chr1 | 1247311 | 21396 | 538 | 2.51 | 0 | 26545 | 9 | 0.03 |
| | chr2 | 106258357 | 30135 | 89 | 0.3 | 0 | 26227 | 4 | 0.02 |
| | chr11 | 5248224 | 44623 | 1 | 0 | 1 | 40363 | 5 | 0.01 |
| | chr4 | 140537415 | 32098 | 320 | 1 | 0 | 40310 | 11 | 0.03 |
| | chr1 | 14704357 | 25687 | 0 | 0 | 1 | 28659 | 1 | 0 |
| | chr12 | 33171137 | 41239 | 12 | 0.03 | 1 | 38487 | 4 | 0.01 |
| | chr10 | 42534687 | 37812 | 1 | 0 | 1 | 31378 | 0 | 0 |
| | chr11 | 24428875 | 3199 | 1 | 0.03 | ND | 1719 | 1 | 0.06 |
| | chr3 | 23606938 | ND | ND | ND | ND | ND | ND | ND |
| | chrX | 53798450 | ND | ND | ND | ND | ND | ND | ND |
| | chr5 | 145526114 | 1663 | 3 | 0.18 | ND | 1658 | 2 | 0.12 |
| | chr7 | 80399197 | ND | ND | ND | ND | ND | ND | ND |
| | chr1 | 199403463 | ND | ND | ND | ND | ND | ND | ND |

| | |
|---|---|
| Aggregate off-target | 6.58 |

For the previously described 8266:20505 ZFN pair, this process revealed 9 active off-target sites with an aggregate activity of 33.3% (Table 1B). For the two new ZFN pairs that targeted the same sequence, 46693:46696 and 46693:46697, this process yielded 11 active off-target sites with an aggregate activity of 21.2% and 10 active off-target sites with an aggregate off-target activity of 6.6% respectively. The decrease in aggregate off-target activity may be due to improved genome-wide specificity as compared to the previously described ZFN pair or simply a reflection of their lower overall activity under these conditions.

The specificity of the new ZFN pairs that target a slightly shifted and extended sequence, 46698:46705 and 46700:46705, allow a more meaningful comparison because they have similar activity to the previously described ZFN pair at the intended CCR5 site. For 46698:46705, four active off-target sites were identified with an aggregate activity of 1.5% (Table 1A). The best results were obtained with 46700:46705 that had three active off-target sites and an aggregate activity of 0.5%. This represents 22-fold and 67-fold decreases in aggregate off-target activity respectively and implies that 46700:46705 is the most specific zinc finger nuclease described in the scientific literature.

Thus, unbiased IDLV screen and confirmed the genome-wide fidelity of ZFNs selected using the multi-reporter assay system as described herein.

The results described herein show that the novel multi-reporter selection system described herein allows for the assay and selection of zinc fingers that able to discriminate between similar targets in vivo.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

1. A genetically modified cell or cell line wherein the modification comprises an insertion and/or deletion at or near any of SEQ ID NOs: 66, 94, 127, 128, or 129 within a hemoglobin beta (HBB) gene.

2. The genetically modified cell of claim 1, wherein the cell is a stem cell.

3. The genetically modified cell of claim 2, wherein the stem cell is a red blood cell (RBC) precursor cell or hematopoietic stem cell.

4. The genetically modified cell of claim 3, wherein the cell is differentiated into a red blood cell (RBC).

5. The genetically modified cell of claim 3, wherein the hematopoietic stem cell is a CD34+ hematopoietic stem cell.

6. A cell descended the cell or cell line according to claim 1.

7. A pharmaceutical composition comprising a genetically modified cell of claim 1.

8. The genetically modified cell of claim 1, wherein the modification is made using a nuclease, the nuclease comprising at least one zinc finger nuclease.

9. The genetically modified cell of claim 8, wherein the nuclease is introduced into the cell as a polynucleotide.

10. The genetically modified cell of claim 1, wherein the insertion comprises integration of a donor polynucleotide encoding a transgene.

11. The genetically modified cell of claim 8, wherein the zinc finger nuclease comprises 4, 5, or 6 zinc finger domains comprising a recognition helix and further wherein the zinc finger proteins comprise the recognition helix regions in the order shown in a single row of Table A.

12. A zinc finger protein comprising the recognition helix regions in the order and sequence shown in Table A.

13. A fusion protein comprising a zinc finger protein of claim 12 and a cleavage domain or cleavage half-domain.

14. The fusion protein of claim 13, wherein the cleavage domain or cleavage half-domain is a wild-type or engineered domain.

15. A polynucleotide encoding a protein of claim 12.

16. An isolated cell comprising one or more proteins according to claim 12.

17. An isolated cell comprising one or more polynucleotides according to claim 15.

18. The cell of claim 17, wherein the cell is a red blood cell precursor cell.

19. The cell of claim 18, wherein the cell is a red blood cell (RBC).

20. A kit comprising a protein according to claim 12.

21. A kit comprising a polynucleotide according to claim 15.

22. A method of altering globin gene expression in a cell, the method comprising:
introducing, into the cell, one or more polynucleotides according to claim 15, under conditions such that the one or more proteins are expressed and expression of the globin gene is altered.

23. The method of claim 22, wherein the proteins increase expression of a beta globin gene.

24. The method of claim 22, further comprising integrating a donor sequence into the genome of the cell.

25. The method of claim 24, wherein the donor sequence comprises a transgene under the control of an endogenous promoter.

26. The method of claim 24, wherein the donor sequence comprises a transgene under the control of an exogenous promoter.

27. A genetically modified cell or cell line wherein the modification comprises an insertion and/or deletion at or near any of SEQ ID NOs: 28-33 or 142 within a CCR5 gene.


SEQUENCE LISTING (215 sequences)

SEQ ID NO: 1 - DNA, 9 bp, Artificial Sequence
  Description of Artificial Sequence Synthetic oligonucleotide
gcgtgggcg

SEQ ID NO: 2 - DNA, 9 bp, Artificial Sequence
  Description of Artificial Sequence Synthetic oligonucleotide
cctgatcct

SEQ ID NO: 3 - DNA, 9 bp, Artificial Sequence
  Description of Artificial Sequence Synthetic oligonucleotide
gcgtgggtg

SEQ ID NO: 4 - DNA, 9 bp, Artificial Sequence
  Description of Artificial Sequence Synthetic oligonucleotide
cctgatcct

SEQ ID NO: 5 - DNA, 9 bp, Artificial Sequence
  Description of Artificial Sequence Synthetic oligonucleotide
gcgtgggcc

SEQ ID NO: 6 - DNA, 9 bp, Artificial Sequence
  Description of Artificial Sequence Synthetic oligonucleotide
gcgtggggg

SEQ ID NO: 7 - DNA, 12 bp, Artificial Sequence
  Description of Artificial Sequence Synthetic oligonucleotide
aaactgcaaaaa

SEQ ID NO: 8 - DNA, 12 bp, Artificial Sequence
  Description of Artificial Sequence Synthetic oligonucleotide
aaactgcaaaag

SEQ ID NO: 9 - DNA, 31 bp, Artificial Sequence
  Description of Artificial Sequence Synthetic oligonucleotide
ggtcatcctcatcctgataaactgcaaaagg

SEQ ID NO: 10 - DNA, 31 bp, Artificial Sequence
  Description of Artificial Sequence Synthetic oligonucleotide
ggtcgtcctcatcttaataaactgcaaaaag

SEQ ID NO: 11 - DNA, 14 bp, Artificial Sequence
  Description of Artificial Sequence Synthetic oligonucleotide
ggatgaggatgacc

SEQ ID NO: 12 - DNA, 14 bp, Artificial Sequence
  Description of Artificial Sequence Synthetic oligonucleotide
taaactgcaaaagg

SEQ ID NO: 13 - DNA, 60 bp, Artificial Sequence
  Description of Artificial Sequence Synthetic oligonucleotide
caacatgctggtcatcctcatcctgataaactgcaaaaggctgaagagcatgactgacat

SEQ ID NO: 14 - DNA, 60 bp, Artificial Sequence
  Description of Artificial Sequence Synthetic oligonucleotide
caacatgctggtcgtcctcatcttaataaactgcaaaaagctgaagtgcttgactgacat

SEQ ID NO: 15 - DNA, 12 bp, Artificial Sequence
  Description of Artificial Sequence Synthetic oligonucleotide
atcaggatgagg

SEQ ID NO: 16 - DNA, 12 bp, Artificial Sequence
  Description of Artificial Sequence Synthetic oligonucleotide
atgaggatgacc

SEQ ID NO: 17 - DNA, 12 bp, Artificial Sequence
  Description of Artificial Sequence Synthetic oligonucleotide
caaaaggctgaa

SEQ ID NO: 18 - DNA, 12 bp, Artificial Sequence
  Description of Artificial Sequence Synthetic oligonucleotide
gctgaagagcat

SEQ ID NO: 19 - DNA, 12 bp, Artificial Sequence
  Description of Artificial Sequence Synthetic oligonucleotide
aaactgcaaaag

SEQ ID NO: 20 - DNA, 361 bp, Artificial Sequence
  Description of Artificial Sequence Synthetic polynucleotide
  modified_base (49)..(50): a, c, t, g, unknown or other
  modified_base (52)..(53): a, c, t, g, unknown or other
  modified_base (55)..(56): a, c, t, g, unknown or other
  modified_base (58)..(59): a, c, t, g, unknown or other
  modified_base (64)..(65): a, c, t, g, unknown or other
  modified_base (67)..(68): a, c, t, g, unknown or other
  modified_base (133)..(134): a, c, t, g, unknown or other
  modified_base (136)..(137): a, c, t, g, unknown or other
  modified_base (139)..(140): a, c, t, g, unknown or other
  modified_base (142)..(143): a, c, t, g, unknown or other
  modified_base (148)..(149): a, c, t, g, unknown or other
  modified_base (151)..(152): a, c, t, g, unknown or other
  modified_base (220)..(221): a, c, t, g, unknown or other
  modified_base (223)..(224): a, c, t, g, unknown or other
  modified_base (226)..(227): a, c, t, g, unknown or other
  modified_base (229)..(230): a, c, t, g, unknown or other
  modified_base (235)..(236): a, c, t, g, unknown or other
  modified_base (238)..(239): a, c, t, g, unknown or other
  modified_base (304)..(305): a, c, t, g, unknown or other
  modified_base (307)..(308): a, c, t, g, unknown or other
  modified_base (310)..(311): a, c, t, g, unknown or other
  modified_base (313)..(314): a, c, t, g, unknown or other
  modified_base (319)..(320): a, c, t, g, unknown or other
  modified_base (322)..(323): a, c, t, g, unknown or other
ggtaccgagcgcccattccagtgtcgaatctgcatgcgtaacttcagtnnsnnsnnsnnsctgnnsnnscacatccgcacccacaccggcgagaagccttttgcctgtgacatttgtgggaggaagtttgccnnsnnsnnsnnsctgnnsnnscataccaaaatccatacaggttcccagaaaccgtttcaatgcaggatatgcatgcgtaacttcagtnnsnnsnnsnnsctgnnsnnscacatccgcacccacaccggcgagaagccttttgcctgtgacatttgtgggaggaagtttgccnnsnnsnnsnnsctgnnsnnscataccaaaatccatttacgtggatcctaagtctaga

SEQ ID NO: 21 - DNA, 361 bp, Artificial Sequence
  Description of Artificial Sequence Synthetic polynucleotide
  modified_base (49)..(50): a, c, t, g, unknown or other
  modified_base (52)..(53): a, c, t, g, unknown or other
  modified_base (55)..(56): a, c, t, g, unknown or other
  modified_base (58)..(59): a, c, t, g, unknown or other
  modified_base (64)..(65): a, c, t, g, unknown or other
  modified_base (67)..(68): a, c, t, g, unknown or other
  modified_base (133)..(134): a, c, t, g, unknown or other
  modified_base (136)..(137): a, c, t, g, unknown or other
  modified_base (139)..(140): a, c, t, g, unknown or other
  modified_base (142)..(143): a, c, t, g, unknown or other
  modified_base (148)..(149): a, c, t, g, unknown or other
  modified_base (151)..(152): a, c, t, g, unknown or other
  modified_base (220)..(221): a, c, t, g, unknown or other
  modified_base (223)..(224): a, c, t, g, unknown or other
  modified_base (226)..(227): a, c, t, g, unknown or other
  modified_base (229)..(230): a, c, t, g, unknown or other
  modified_base (235)..(236): a, c, t, g, unknown or other
  modified_base (238)..(239): a, c, t, g, unknown or other
  modified_base (304)..(305): a, c, t, g, unknown or other
  modified_base (307)..(308): a, c, t, g, unknown or other
  modified_base (310)..(311): a, c, t, g, unknown or other
  modified_base (313)..(314): a, c, t, g, unknown or other
  modified_base (319)..(320): a, c, t, g, unknown or other
  modified_base (322)..(323): a, c, t, g, unknown or other
ggtaccgagcgcccatttcaatgcaggatatgcatgcgtaacttcagtnnsnnsnnsnnsctgnnsnnscacatccgcacccacaccggcgagaagccttttgcctgtgacatttgtgggaggaagtttgccnnsnnsnnsnnsctgnnsnnscataccaaaatccataccggcagccagaagccatttcagtgccgcatttgcatgcgtaacttcagtnnsnnsnnsnnsctgnnsnnscacatccgcacccacaccggcgagaagccttttgcctgtgacatttgtgggaggaagtttgccnnsnnsnnsnnsctgnnsnnscataccaaaatccatttacgtggatcctaagtctaga

SEQ ID NO: 22 - DNA, 70 bp, Artificial Sequence
  Description of Artificial Sequence Synthetic oligonucleotide
  modified_base (25)..(46): a, c, t or g
cagggatccatgcactgtacgcccnnnnnnnnnnnnnnnnnnnnnngggccacttgactgcggatcctgg

SEQ ID NO: 23 - DNA, 21 bp, Artificial Sequence
  Description of Artificial Sequence Synthetic primer
ccaggatccgcagtcaagtgg

SEQ ID NO: 24 - DNA, 21 bp, Artificial Sequence
  Description of Artificial Sequence Synthetic primer
cagggatccatgcactgtacg

SEQ ID NO: 25 - DNA, 20 bp, Artificial Sequence
  Description of Artificial Sequence Synthetic primer
acacgacgctcttccgatct

SEQ ID NO: 26 - DNA, 18 bp, Artificial Sequence
  Description of Artificial Sequence Synthetic primer
gacgtgtgctcttccgat

SEQ ID NO: 27 - DNA, 12 bp, Artificial Sequence
  Description of Artificial Sequence Synthetic oligonucleotide
aaactgcaaaag

SEQ ID NO: 28 - DNA, 12 bp, Artificial Sequence
  Description of Artificial Sequence Synthetic oligonucleotide
gatgaggatgac

SEQ ID NO: 29 - DNA, 12 bp, Artificial Sequence
  Description of Artificial Sequence Synthetic oligonucleotide
aaactgcaaaag

SEQ ID NO: 30 - DNA, 28 bp, Artificial Sequence
  Description of Artificial Sequence Synthetic oligonucleotide
aggatgaggatgaccagcatgttgttgc

SEQ ID NO: 31 - DNA, 28 bp, Artificial Sequence
  Description of Artificial Sequence Synthetic oligonucleotide
ataaactgcaaaaggctgaagagcatga

SEQ ID NO: 32 - DNA, 28 bp, Artificial Sequence
  Description of Artificial Sequence Synthetic oligonucleotide
ttatcaggatgaggatgaccagcatgtt

SEQ ID NO: 33 - DNA, 28 bp, Artificial Sequence
  Description of Artificial Sequence Synthetic oligonucleotide
tgcaaaaggctgaagagcatgactgaca

SEQ ID NO: 34 - protein, 7 aa, Artificial Sequence
  Description of Artificial Sequence Synthetic peptide
DRSNLSR

SEQ ID NO: 35 - protein, 7 aa, Artificial Sequence
  Description of Artificial Sequence Synthetic peptide
ISSNLNS

SEQ ID NO: 36 - protein, 7 aa, Artificial Sequence
  Description of Artificial Sequence Synthetic peptide
RSDNLAR

SEQ ID NO: 37 - protein, 7 aa, Artificial Sequence
  Description of Artificial Sequence Synthetic peptide
TSGNLTR

SEQ ID NO: 38 - protein, 7 aa, Artificial Sequence
  Description of Artificial Sequence Synthetic peptide
RSDNLSV

SEQ ID NO: 39 - protein, 7 aa, Artificial Sequence
  Description of Artificial Sequence Synthetic peptide
QKINLQV

SEQ ID NO: 40 - protein, 7 aa, Artificial Sequence
  Description of Artificial Sequence Synthetic peptide
RSDVLSE

SEQ ID NO: 41 - protein, 7 aa, Artificial Sequence
  Description of Artificial Sequence Synthetic peptide
QRNHRTT

SEQ ID NO: 42 - protein, 7 aa, Artificial Sequence
  Description of Artificial Sequence Synthetic peptide
DQSNLTR

SEQ ID NO: 43 - protein, 7 aa, Artificial Sequence
  Description of Artificial Sequence Synthetic peptide
APSNLWR

SEQ ID NO: 44 - protein, 7 aa, Artificial Sequence
  Description of Artificial Sequence Synthetic peptide
TLYNLTR

SEQ ID NO: 45 - protein, 7 aa, Artificial Sequence
  Description of Artificial Sequence Synthetic peptide
FLGNLTR

SEQ ID NO: 46 - protein, 7 aa, Artificial Sequence
  Description of Artificial Sequence Synthetic peptide
TKWNLTT

SEQ ID NO: 47 - protein, 7 aa, Artificial Sequence
  Description of Artificial Sequence Synthetic peptide
QKINLTA

SEQ ID NO: 48 - protein, 7 aa, Artificial Sequence
  Description of Artificial Sequence Synthetic peptide
RKWVLDC

SEQ ID NO: 49 - protein, 7 aa, Artificial Sequence
  Description of Artificial Sequence Synthetic peptide
NPGSLHN

SEQ ID NO: 50 - protein, 7 aa, Artificial Sequence
  Description of Artificial Sequence Synthetic peptide
RKSTLND

SEQ ID NO: 51 - protein, 7 aa, Artificial Sequence
  Description of Artificial Sequence Synthetic peptide
QKGNLNQ

SEQ ID NO: 52 - protein, 7 aa, Artificial Sequence
  Description of Artificial Sequence Synthetic peptide
RKAHLVN

SEQ ID NO: 53 - protein, 7 aa, Artificial Sequence
  Description of Artificial Sequence Synthetic peptide
WQSGLCN

SEQ ID NO: 54 - protein, 7 aa, Artificial Sequence
  Description of Artificial Sequence Synthetic peptide
RKSHLVD

SEQ ID NO: 55 - protein, 7 aa, Artificial Sequence
  Description of Artificial Sequence Synthetic peptide
SASGLCH

SEQ ID NO: 56 - protein, 7 aa, Artificial Sequence
  Description of Artificial Sequence Synthetic peptide
TKQNLTH

SEQ ID NO: 57 - protein, 7 aa, Artificial Sequence
  Description of Artificial Sequence Synthetic peptide
SLFNLKR

SEQ ID NO: 58 - protein, 7 aa, Artificial Sequence
  Description of Artificial Sequence Synthetic peptide
QLCNLIR

SEQ ID NO: 59 - protein, 7 aa, Artificial Sequence
  Description of Artificial Sequence Synthetic peptide
LKSTLVN

SEQ ID NO: 60 - protein, 7 aa, Artificial Sequence
  Description of Artificial Sequence Synthetic peptide
RKDNLKS

SEQ ID NO: 61 - protein, 7 aa, Artificial Sequence
  Description of Artificial Sequence Synthetic peptide
QKINLVN

SEQ ID NO: 62 - protein, 7 aa, Artificial Sequence
  Description of Artificial Sequence Synthetic peptide
DPRSLVN

SEQ ID NO: 63 - protein, 7 aa, Artificial Sequence
  Description of Artificial Sequence Synthetic peptide
ARNGLWQ

SEQ ID NO: 64 - protein, 7 aa, Artificial Sequence
  Description of Artificial Sequence Synthetic peptide
RKDNLRT

SEQ ID NO: 65 - protein, 7 aa, Artificial Sequence
  Description of Artificial Sequence Synthetic peptide
QKGNLNS

SEQ ID NO: 66 - DNA, 12 bp, Artificial Sequence
  Description of Artificial Sequence Synthetic oligonucleotide
tcccgtcattgc

SEQ ID NO: 67 - protein, 7 aa, Artificial Sequence
  Description of Artificial Sequence Synthetic peptide
RKQCLQR

SEQ ID NO: 68 - protein, 7 aa, Artificial Sequence
  Description of Artificial Sequence Synthetic peptide
WPNSLKA

SEQ ID NO: 69 - protein, 7 aa, Artificial Sequence
  Description of Artificial Sequence Synthetic peptide
DQTNLRK

SEQ ID NO: 70 - protein, 7 aa, Artificial Sequence
  Description of Artificial Sequence Synthetic peptide
HKHHLSQ

SEQ ID NO: 71 - protein, 7 aa, Artificial Sequence
  Description of Artificial Sequence Synthetic peptide
RRQCLQR

SEQ ID NO: 72 - protein, 7 aa, Artificial Sequence
  Description of Artificial Sequence Synthetic peptide
DRSNLTK

SEQ ID NO: 73 - protein, 7 aa, Artificial Sequence
  Description of Artificial Sequence Synthetic peptide
HNHHLTQ

SEQ ID NO: 74 - protein, 7 aa, Artificial Sequence
  Description of Artificial Sequence Synthetic peptide
RRQCLRR

SEQ ID NO: 75 - protein, 7 aa, Artificial Sequence
  Description of Artificial Sequence Synthetic peptide
DRANLIK

SEQ ID NO: 76 - protein, 7 aa, Artificial Sequence
  Description of Artificial Sequence Synthetic peptide
HKHHLTE

SEQ ID NO: 77 - protein, 7 aa, Artificial Sequence
  Description of Artificial Sequence Synthetic peptide
DRTNLIK

SEQ ID NO: 78 - protein, 7 aa, Artificial Sequence
  Description of Artificial Sequence Synthetic peptide
MKHHLSS

SEQ ID NO: 79 - protein, 7 aa, Artificial Sequence
  Description of Artificial Sequence Synthetic peptide
RRQCLTR

SEQ ID NO: 80 - protein, 7 aa, Artificial Sequence
  Description of Artificial Sequence Synthetic peptide
WANSLRA

SEQ ID NO: 81 - protein, 7 aa, Artificial Sequence
  Description of Artificial Sequence Synthetic peptide
LKGNLKK

SEQ ID NO: 82 - protein, 7 aa, Artificial Sequence
  Description of Artificial Sequence Synthetic peptide
HKHHLTD

SEQ ID NO: 83 - protein, 7 aa, Artificial Sequence
  Description of Artificial Sequence Synthetic peptide
RKQDLQR

SEQ ID NO: 84 - protein, 7 aa, Artificial Sequence
  Description of Artificial Sequence Synthetic peptide
WPNSLRY

SEQ ID NO: 85 - protein, 7 aa, Artificial Sequence
  Description of Artificial Sequence Synthetic peptide
DRSNLLK

SEQ ID NO: 86 - protein, 7 aa, Artificial Sequence
  Description of Artificial Sequence Synthetic peptide
MKHHLKE

SEQ ID NO: 87 - protein, 7 aa, Artificial Sequence
  Description of Artificial Sequence Synthetic peptide
DRTNLLK

SEQ ID NO: 88 - protein, 7 aa, Artificial Sequence
  Description of Artificial Sequence Synthetic peptide
LNHHLTD

SEQ ID NO: 89 - protein, 7 aa, Artificial Sequence
  Description of Artificial Sequence Synthetic peptide
RNQCLQR

SEQ ID NO: 90 - protein, 7 aa, Artificial Sequence
  Description of Artificial Sequence Synthetic peptide
LRGNLNK

SEQ ID NO: 91 - protein, 7 aa, Artificial Sequence
  Description of Artificial Sequence Synthetic peptide
WANSLRY

SEQ ID NO: 92 - protein, 7 aa, Artificial Sequence
  Description of Artificial Sequence Synthetic peptide
DRANLLK

SEQ ID NO: 93 - protein, 7 aa, Artificial Sequence
  Description of Artificial Sequence Synthetic peptide
MKQHLTS

SEQ ID NO: 94 - DNA, 12 bp, Artificial Sequence
  Description of Artificial Sequence Synthetic oligonucleotide
cattgccgtctg

SEQ ID NO: 95 - protein, 7 aa, Artificial Sequence
  Description of Artificial Sequence Synthetic peptide
DRTNLTS

SEQ ID NO: 96 - protein, 7 aa, Artificial Sequence
  Description of Artificial Sequence Synthetic peptide
SHHHLTE

SEQ ID NO: 97 - protein, 7 aa, Artificial Sequence
  Description of Artificial Sequence Synthetic peptide
WPNSLKY

SEQ ID NO: 98 - protein, 7 aa, Artificial Sequence
  Description of Artificial Sequence Synthetic peptide
DQSALIR

SEQ ID NO: 99 - protein, 7 aa, Artificial Sequence
  Description of Artificial Sequence Synthetic peptide
LKGNLIK

SEQ ID NO: 100 - protein, 7 aa, Artificial Sequence
  Description of Artificial Sequence Synthetic peptide
SSWHLKE

SEQ ID NO: 101 - protein, 7 aa, Artificial Sequence
  Description of Artificial Sequence Synthetic peptide
WAYMLRR

SEQ ID NO: 102 - protein, 7 aa, Artificial Sequence
  Description of Artificial Sequence Synthetic peptide
LKGNLTK

SEQ ID NO: 103 - protein, 7 aa, Artificial Sequence
  Description of Artificial Sequence Synthetic peptide
SRHHLTE

SEQ ID NO: 104 - protein, 7 aa, Artificial Sequence
  Description of Artificial Sequence Synthetic peptide
WHNSLKY

SEQ ID NO: 105 - protein, 7 aa, Artificial Sequence
  Description of Artificial Sequence Synthetic peptide
DRSALIR

SEQ ID NO: 106 - protein, 7 aa, Artificial Sequence
  Description of Artificial Sequence Synthetic peptide
DRTNLTK

SEQ ID NO: 107 - protein, 7 aa, Artificial Sequence
  Description of Artificial Sequence Synthetic peptide
HKHHLVE

SEQ ID NO: 108 - protein, 7 aa, Artificial Sequence
  Description of Artificial Sequence Synthetic peptide
WKSSLKA

SEQ ID NO: 109 - protein, 7 aa, Artificial Sequence
  Description of Artificial Sequence Synthetic peptide
DKSSLIR

SEQ ID NO: 110 - protein, 7 aa, Artificial Sequence
  Description of Artificial Sequence Synthetic peptide
MQHHLTE

SEQ ID NO: 111 - protein, 7 aa, Artificial Sequence
  Description of Artificial Sequence Synthetic peptide
DRSSLLR

SEQ ID NO: 112 - protein, 7 aa, Artificial Sequence
  Description of Artificial Sequence Synthetic peptide
NEWHLNE

SEQ ID NO: 113 - protein, 7 aa, Artificial Sequence
  Description of Artificial Sequence Synthetic peptide
WANSLKY

SEQ ID NO: 114 - protein, 7 aa, Artificial Sequence
  Description of Artificial Sequence Synthetic peptide
DGSALIR

SEQ ID NO: 115 - protein, 7 aa, Artificial Sequence
  Description of Artificial Sequence Synthetic peptide
LKGNLLK

SEQ ID NO: 116 - protein, 7 aa, Artificial Sequence
  Description of Artificial Sequence Synthetic peptide
MKHHLTE

SEQ ID NO: 117 - protein, 7 aa, Artificial Sequence
  Description of Artificial Sequence Synthetic peptide
DRSALLR

SEQ ID NO: 118 - protein, 7 aa, Artificial Sequence
  Description of Artificial Sequence Synthetic peptide
SKRSLTE

SEQ ID NO: 119 - protein, 7 aa, Artificial Sequence
  Description of Artificial Sequence Synthetic peptide
HKSHLAD

SEQ ID NO: 120 - protein, 7 aa, Artificial Sequence
  Description of Artificial Sequence Synthetic peptide
FNYMLRR

SEQ ID NO: 121 - protein, 7 aa, Artificial Sequence
  Description of Artificial Sequence Synthetic peptide
SSWHLRE

SEQ ID NO: 122 - protein, 7 aa, Artificial Sequence
  Description of Artificial Sequence Synthetic peptide
SRNGLTY

SEQ ID NO: 123 - protein, 7 aa, Artificial Sequence
  Description of Artificial Sequence Synthetic peptide
LNGNLKK

SEQ ID NO: 124 - protein, 7 aa, Artificial Sequence
  Description of Artificial Sequence Synthetic peptide
HKHHLMD

SEQ ID NO: 125 - protein, 7 aa, Artificial Sequence
  Description of Artificial Sequence Synthetic peptide
WYNSLRY

SEQ ID NO: 126 - protein, 7 aa, Artificial Sequence
  Description of Artificial Sequence Synthetic peptide
DKSSLLR

SEQ ID NO: 127 - DNA, 28 bp, Artificial Sequence
  Description of Artificial Sequence Synthetic oligonucleotide
tccacaggagtcagatgcaccatggtgt

SEQ ID NO: 128 - DNA, 28 bp, Artificial Sequence
  Description of Artificial Sequence Synthetic oligonucleotide
agtctgccgttactgccctgtggggcaa

SEQ ID NO: 129 - DNA, 28 bp, Artificial Sequence
  Description of Artificial Sequence Synthetic oligonucleotide
aagtctgccgttactgccctgtggggca

SEQ ID NO: 130 - protein, 7 aa, Artificial Sequence
  Description of Artificial Sequence Synthetic peptide
TKWNLTQ

SEQ ID NO: 131 - protein, 7 aa, Artificial Sequence
  Description of Artificial Sequence Synthetic peptide
FKSNLTN

SEQ ID NO: 132 - protein, 7 aa, Artificial Sequence
  Description of Artificial Sequence Synthetic peptide
RKAHLVN

SEQ ID NO: 133 - protein, 7 aa, Artificial Sequence
  Description of Artificial Sequence Synthetic peptide
DRANLIH

SEQ ID NO: 134 - protein, 7 aa, Artificial Sequence
  Description of Artificial Sequence Synthetic peptide
DRSNLRA

SEQ ID NO: 135 - protein, 7 aa, Artificial Sequence
  Description of Artificial Sequence Synthetic peptide
RKFTLTN

SEQ ID NO: 136 - DNA, 43 bp, Artificial Sequence
  Description of Artificial Sequence Synthetic oligonucleotide
ggtgcatctgactcctgtggagaagtctgccgttactgccctg

SEQ ID NO: 137 - protein, 7 aa, Artificial Sequence
  Description of Artificial Sequence Synthetic peptide
TKWNLDT

SEQ ID NO: 138 - protein, 7 aa, Artificial Sequence
  Description of Artificial Sequence Synthetic peptide
RASTLWH

SEQ ID NO: 139 - protein, 7 aa, Artificial Sequence
  Description of Artificial Sequence Synthetic peptide
RKSTLVE

SEQ ID NO: 140 - protein, 7 aa, Artificial Sequence
  Description of Artificial Sequence Synthetic peptide
QRGNLKT

SEQ ID NO: 141 - DNA, 23 bp, Artificial Sequence
  Description of Artificial Sequence Synthetic oligonucleotide
ataaactgcaaaaggctgaagag

SEQ ID NO: 142 - DNA, 23 bp, Artificial Sequence
  Description of Artificial Sequence Synthetic oligonucleotide
aggatgaggatgaccagcatgtt

SEQ ID NO: 143 - protein, 7 aa, Artificial Sequence
  Description of Artificial Sequence Synthetic peptide
DRSNLLR

SEQ ID NO: 144 - protein, 7 aa, Artificial Sequence
  Description of Artificial Sequence Synthetic peptide
FLGNLRR

SEQ ID NO: 145 - protein, 7 aa, Artificial Sequence
  Description of Artificial Sequence Synthetic peptide
TQFNLER

SEQ ID NO: 146 - protein, 7 aa, Artificial Sequence
  Description of Artificial Sequence Synthetic peptide
MRANLRR

SEQ ID NO: 147 - protein, 7 aa, Artificial Sequence
  Description of Artificial Sequence Synthetic peptide
WQANLLR

SEQ ID NO: 148 - protein, 7 aa, Artificial Sequence
  Description of Artificial Sequence Synthetic peptide
FASNLIR

SEQ ID NO: 149 - protein, 7 aa, Artificial Sequence
  Description of Artificial Sequence Synthetic peptide
TLWSLTR

SEQ ID NO: 150 - protein, 7 aa, Artificial Sequence
  Description of Artificial Sequence Synthetic peptide
TKQNLQR

SEQ ID NO: 151 - protein, 7 aa, Artificial Sequence
  Description of Artificial Sequence Synthetic peptide
RKDNLTQ

SEQ ID NO: 152 - protein, 7 aa, Artificial Sequence
  Description of Artificial Sequence Synthetic peptide
RKAHLVD

SEQ ID NO: 153 - protein, 7 aa, Artificial Sequence
  Description of Artificial Sequence Synthetic peptide
ARAGLWQ

SEQ ID NO: 154 - protein, 7 aa, Artificial Sequence
  Description of Artificial Sequence Synthetic peptide
RKANLYN

SEQ ID NO: 155 - protein, 7 aa, Artificial Sequence
  Description of Artificial Sequence Synthetic peptide
SQSGLCH

SEQ ID NO: 156 - protein, 7 aa, Artificial Sequence
  Description of Artificial Sequence Synthetic peptide
EKRGLLN

SEQ ID NO: 157 - protein, 7 aa, Artificial Sequence
  Description of Artificial Sequence Synthetic peptide
SGAGLWQ

SEQ ID NO: 158 - protein, 7 aa, Artificial Sequence
  Description of Artificial Sequence Synthetic peptide
AQSNLLR

SEQ ID NO: 159 - protein, 7 aa, Artificial Sequence
  Description of Artificial Sequence Synthetic peptide
RKPDLVR

SEQ ID NO: 160 - protein, 7 aa, Artificial Sequence
  Description of Artificial Sequence Synthetic peptide
RKDNLRD

SEQ ID NO: 161 - protein, 7 aa, Artificial Sequence
  Description of Artificial Sequence Synthetic peptide
QKINLNQ

SEQ ID NO: 162 - protein, 7 aa, Artificial Sequence
  Description of Artificial Sequence Synthetic peptide
TKQNLQT

SEQ ID NO: 163 - protein, 7 aa, Artificial Sequence
  Description of Artificial Sequence Synthetic peptide
TLFNLTR

SEQ ID NO: 164 - DNA, 12 bp, Artificial Sequence
  Description of Artificial Sequence Synthetic oligonucleotide
agtcagatgcac

SEQ ID NO: 165 - DNA, 12 bp, Artificial Sequence
  Description of Artificial Sequence Synthetic oligonucleotide
cacaggagtcag

SEQ ID NO: 166 - protein, 7 aa, Artificial Sequence
  Description of Artificial Sequence Synthetic peptide
DRSNLRA

SEQ ID NO: 167 - protein, 7 aa, Artificial Sequence
  Description of Artificial Sequence Synthetic peptide
RNSTLIE

SEQ ID NO: 168 - protein, 7 aa, Artificial Sequence
  Description of Artificial Sequence Synthetic peptide
RKDNLTQ

SEQ ID NO: 169 - protein, 7 aa, Artificial Sequence
  Description of Artificial Sequence Synthetic peptide
FKSNLTS

SEQ ID NO: 170 - protein, 7 aa, Artificial Sequence
  Description of Artificial Sequence Synthetic peptide
RKFTLTN

SEQ ID NO: 171 - protein, 7 aa, Artificial Sequence
  Description of Artificial Sequence Synthetic peptide
RKGNLKE

SEQ ID NO: 172 - protein, 7 aa, Artificial Sequence
  Description of Artificial Sequence Synthetic peptide
FHSNLIA

SEQ ID NO: 173 - protein, 7 aa, Artificial Sequence
  Description of Artificial Sequence Synthetic peptide
RKSTLRL

SEQ ID NO: 174 - protein, 7 aa, Artificial Sequence
  Description of Artificial Sequence Synthetic peptide
RKGNLNS

SEQ ID NO: 175 - protein, 7 aa, Artificial Sequence
  Description of Artificial Sequence Synthetic peptide
WKSNLTS

SEQ ID NO: 176 - protein, 7 aa, Artificial Sequence
  Description of Artificial Sequence Synthetic peptide
LKGNLRQ

SEQ ID NO: 177 - protein, 7 aa, Artificial Sequence
  Description of Artificial Sequence Synthetic peptide
RKANLRD

SEQ ID NO: 178 - protein, 7 aa, Artificial Sequence
  Description of Artificial Sequence Synthetic peptide
DRSNLRH

SEQ ID NO: 179 - protein, 7 aa, Artificial Sequence
  Description of Artificial Sequence Synthetic peptide
RKGNLKD

SEQ ID NO: 180 - protein, 7 aa, Artificial Sequence
  Description of Artificial Sequence Synthetic peptide
MKHHLTD

SEQ ID NO: 181 - protein, 7 aa, Artificial Sequence
  Description of Artificial Sequence Synthetic peptide
RKFVLTN

SEQ ID NO: 182 - protein, 7 aa, Artificial Sequence
  Description of Artificial Sequence Synthetic peptide
TKWNLTQ

SEQ ID NO: 183 - protein, 7 aa, Artificial Sequence
  Description of Artificial Sequence Synthetic peptide
WKSNLTN

SEQ ID NO: 184 - protein, 7 aa, Artificial Sequence
  Description of Artificial Sequence Synthetic peptide
FQSNLTN

SEQ ID NO: 185 - protein, 7 aa, Artificial Sequence
  Description of Artificial Sequence Synthetic peptide
YKRSLVD

SEQ ID NO: 186 - protein, 7 aa, Artificial Sequence
  Description of Artificial Sequence Synthetic peptide
RKSTLIE

SEQ ID NO: 187 - protein, 7 aa, Artificial Sequence
  Description of Artificial Sequence Synthetic peptide
MKHHLTS

SEQ ID NO: 188 - protein, 7 aa, Artificial Sequence
  Description of Artificial Sequence Synthetic peptide
RKDTLVT

SEQ ID NO: 189 - protein, 7 aa, Artificial Sequence
  Description of Artificial Sequence Synthetic peptide
DRSNLIA

SEQ ID NO: 190 - protein, 7 aa, Artificial Sequence
  Description of Artificial Sequence Synthetic peptide
RKANLND

SEQ ID NO: 191 - protein, 7 aa, Artificial Sequence
  Description of Artificial Sequence Synthetic peptide
WKHVLTN

SEQ ID NO: 192 - protein, 7 aa, Artificial Sequence
  Description of Artificial Sequence Synthetic peptide
RKDHLVD

SEQ ID NO: 193 - protein, 7 aa, Artificial Sequence
  Description of Artificial Sequence Synthetic peptide
HRSNLIH

SEQ ID NO: 194 - protein, 7 aa, Artificial Sequence
  Description of Artificial Sequence Synthetic peptide
LNQHLTN

SEQ ID NO: 195 - protein, 7 aa, Artificial Sequence
  Description of Artificial Sequence Synthetic peptide
RKDHLVN

SEQ ID NO: 196 - protein, 7 aa, Artificial Sequence
  Description of Artificial Sequence Synthetic peptide
HQSNLIH

SEQ ID NO: 197 - protein, 7 aa, Artificial Sequence
  Description of Artificial Sequence Synthetic peptide
WQSNLTE

SEQ ID NO: 198 - protein, 7 aa, Artificial Sequence
  Description of Artificial Sequence Synthetic peptide
RKAHLYN

SEQ ID NO: 199 - protein, 7 aa, Artificial Sequence
  Description of Artificial Sequence Synthetic peptide
DQANLRH

SEQ ID NO: 200 - protein, 7 aa, Artificial Sequence
  Description of Artificial Sequence Synthetic peptide
TKWNLTT

SEQ ID NO: 201 - protein, 7 aa, Artificial Sequence
  Description of Artificial Sequence Synthetic peptide
RKSHLVD

SEQ ID NO: 202 - protein, 7 aa, Artificial Sequence
  Description of Artificial Sequence Synthetic peptide
CKPNLLS

SEQ ID NO: 203 - protein, 7 aa, Artificial Sequence
  Description of Artificial Sequence Synthetic peptide
LNHHLTQ

SEQ ID NO: 204 - protein, 7 aa, Artificial Sequence
  Description of Artificial Sequence Synthetic peptide
LKHHLTN

SEQ ID NO: 205 - protein, 7 aa, Artificial Sequence
  Description of Artificial Sequence Synthetic peptide
HQANLIH

SEQ ID NO: 206 - DNA, 12 bp, Artificial Sequence
  Description of Artificial Sequence Synthetic oligonucleotide
aaactgcaaaag

SEQ ID NO: 207 - DNA, 18 bp, Artificial Sequence
  Description of Artificial Sequence Synthetic oligonucleotide
caaaaggctgaagagcat

SEQ ID NO: 208 - DNA, 44 bp, Artificial Sequence
  Description of Artificial Sequence Synthetic oligonucleotide
tggtcatcctcatcctgataaactgcaaaaggctgaagagcatg

SEQ ID NO: 209 - DNA, 18 bp, Artificial Sequence
  Description of Artificial Sequence Synthetic oligonucleotide
atcaggatgaggatgacc

SEQ ID NO: 210 - DNA, 60 bp, Artificial Sequence; identical to SEQ ID NO: 13
  Description of Artificial Sequence Synthetic oligonucleotide

SEQ ID NO: 211 - DNA, 60 bp, Artificial Sequence; identical to SEQ ID NO: 14
  Description of Artificial Sequence Synthetic oligonucleotide

SEQ ID NO: 212 - DNA, 18 bp, Artificial Sequence
  Description of Artificial Sequence Synthetic oligonucleotide
gtctgccgttactgccct

SEQ ID NO: 213 - DNA, 18 bp, Artificial Sequence
  Description of Artificial Sequence Synthetic oligonucleotide
cacaggagtcagatgcac

SEQ ID NO: 214 - DNA, 12 bp, Artificial Sequence
  Description of Artificial Sequence Synthetic oligonucleotide
cgttactgccct

SEQ ID NO: 215 - DNA, 12 bp, Artificial Sequence
  Description of Artificial Sequence Synthetic oligonucleotide
gtctgccgttac